(19) United States
(12) Patent Application Publication
(10) Pub. No.: US 2023/0101926 A1
(43) Pub. Date: Mar. 30, 2023

(54) Bone Implant

(71) Applicant: BEN ZION KARMON, PETACH-TIKVA (IL)

(72) Inventor: BEN ZION KARMON, PETACH-TIKVA (IL)

(21) Appl. No.: 17/802,982

(22) PCT Filed: Mar. 2, 2021
(86) PCT No.: PCT/IL21/50233

§ 371 Date: Aug. 29, 2022

(30) Foreign Application Priority Data
Mar. 3, 2020 (IL) 273038

(51) Int. Cl. A61B 5/00; A61B 5/145; A61B 5/1459; A61C 8/00

(52) U.S. Cl. CPC A61B 5/686; A61B 5/1451; A61B 5/1459; A61B 5/6882; A61B 5/14546; A61B 5/14532; A61B 5/6878; A61C 8/0093; A61C 8/0012; A61B 2560/0214

(57) ABSTRACT

Devices and methods to perform measurements of body analytes, like glucose, using a bone implant and/or a dental implant and/or a jawbone implant and an analyte measuring component. The dental implant can have a protruding component that protrudes to the oral cavity for many years and therefore enable long-term handling of the analyte measuring component and long-term monitoring of the analyte. The new device enables for example, to replace at least part of the analyte measuring component, to replace materials of the analyte measuring component and the energy source of the device without surgery.

### FIELD AND BACKGROUND OF THE INVENTION

The present invention relates to devices that can be inserted into the human body to monitor biological parameters and/or analytes and methods for monitoring biological parameters and/or analytes.

Implantable sensors for monitoring biological parameters and/or agents and/or analytes in the human body have been under development for many years. One example is the glucose sensor that promises to provide diabetic patients with improved monitoring of blood sugar levels in order to tailor insulin treatment to alleviate the symptoms and long-term damaging effects of diabetes mellitus. In one form, an implantable electrochemical glucose sensor utilizes an enzyme such as glucose oxidase (GO) to catalytically convert glucose to gluconic acid with the simultaneous consumption of oxygen, which is detected with current measuring electrodes. Such electrodes are commonly fabricated from the platinum-family noble metals, because of such metals' catalytic properties and resistance to corrosion. See, for example, U.S. Pat. No. 4,890,620 granted Jan. 2, 1990 to David A. Gough, the entire disclosure of which is hereby incorporated by reference.

There are many technical challenges in designing a commercially viable implantable sensor that will meet medical device regulatory and performance requirements. First and foremost, it must be safe, accurate and reliable. Fabrication techniques developed in the micro-electronics industry along with specialized electrode energization and signal processing techniques offer the potential to solve many of these problems.

See, for example, U.S. Pat. No. 6,516,808 granted to Joseph H. Schulman, the entire disclosure of which is hereby incorporated by reference.

See, for example, U.S. Application 20110137142 to Lucisano Joseph Y., the entire disclosure of which is hereby incorporated by reference.

See, for example, U.S. Pat. No. 5,487,855 of Moeggenborg, et al. assigned to Nalco Chemical Company, the entire disclosure of which is hereby incorporated by reference.

See, for example, U.S. Application 20140309510 to Lucisano Joseph Y. et al., the entire disclosure of which is hereby incorporated by reference.

There are also implantable optical devices and other techniques for sensing glucose.

See, for example, U.S. Patent Application Publication 20070066877 to Arnold et al., the entire disclosure of which is hereby incorporated by reference.

See, for example, PCT Patent Publications WO 2006/006166, PCT/IL2005/000743, PCT/IL2007/000399, U.S. Provisional Patent Applications 60/588,211, 60/658,716, 60/786,532 and U.S. patent applications 20100160749, 20110251471, 20120059232 all to Gross et al., the entire disclosure of which is hereby incorporated by reference.

See, for example, U.S. Application 20130006069, to Gil Tamir et al., the entire disclosures of which is hereby incorporated by reference.

See, for example, U.S. Application 20150343093, to Hyman Tehila et al., the entire disclosures of which is hereby incorporated by reference.

There are also devices for sensing glucose using polarimetry techniques known in the art. See, for example U.S. Pat. Nos. 5,209,231, 6,188,477; 6,577,393, the entire disclosures of which are hereby incorporated by reference.

There is an implanted sensor under development that measure glucose and/or other analytes by changes of osmotic pressure. See for example U.S. Applications 20060173252, 20100084333, 20100223981 and 20200196917 to Lifecare AS from Norway, the entire disclosures of which are hereby incorporated by reference.

The present non-invasive solutions (not requiring pricking for every measurement) are not accurate. There are some devices that include a needle, which is inserted once inside the body. However, due to the risk of infection, this needle needs to be replaced every several days. Some of these devices are also connected to insulin pumps. See for example U.S. Applications No. 20190282143, 20180338282, 20180055423, 20170261491, 20160302701, 20160198988, 20160183856, 20160183855, 20160183799, 20140303465, 20140278189, 20140275900, 20140266785, 20140257065, 20140213869, 20140188402, 20140184422, 20140182350, 20140180049, 20140148667, 20140148666, 20140142405, 20140128803, 20140128701, 20140118166, 20140118138, 20140114161, 20140114159, 20140114157, 20140114156, 20140096264, 20140046158, 20140012510, 20140012117, 20140005508, 20130321425, 20130310666, 20130267813, 20130267811, 20130253418, 20130245981, 20040045879, 20040011671, 20030217966, and 20030023317 assigned to DexCom Inc. the entire disclosures of which are hereby incorporated by reference.

See for example U.S. patent applications No. 20190274597, 20190216374, 20190209059, 20190167166, 20190159734, 20190151541, 20190150809, 20190069823, 20190069819, 20190056385, 20190056384, 20190056304, 20190054466, 20190035493, 20190033331, 20190033305, 20190024130, 20190014807, 20190004041, 20180321265, 20180321264, 20180304011, 20180235930, 20180235524, 20180220959, 20180217135, 20180214029, 20180213834, 20180202962, 20180169121, 20180164331, 20180161354, 20180161353, 20180161352, 20180161351, 20180140622, 20180133235, 20180128767, 20180104694, 20180104267, 20180095067, 20180085343, assigned to Abbott Diabetes Care Inc. the entire disclosures of which are hereby incorporated by reference.

See for example U.S. patent applications No. 20190274600, 20190241926, 20190239778, 20180325436, 20180311383, 20180263511, 20180116598, 20170315077, 20170290546, 20170290535, 20170290534, 20170290512, 20170246367, 20170172471, 20170000936, 20160228042, 20160129203, 20160106911, 20160051749, 20150331419, 20150316499, 20150306292, 20150238673, 20150231387, 20150144542, 20140336631, 20140278168, 20140217030, 20140217029, 20140217027, 20140217020, 20140216250, 20140190891, 20140190885, 20140190876, 20140171942 assigned to Medtronic MiniMed, Inc. the entire disclosures of which are hereby incorporated by reference.

To solve this problem of infection there are attempts to develop fully implanted sensors that some of them are connected to insulin pumps.

See for example, U.S. patent applications No. 20190239784, 20190159708, 20190159704, 20190142345, 20190142314, 20190125969, 20190121506, 20190094233, 20190079009, 20190076022, 20190046095, 20190046090, 20190015021, 20180368685, 20180360356, 20180357200, 20180353113, 20180279923, 20180228408, 20180223050, 20180184953, 20180177396, 20180160974, 20180146885, 20180137070, 20180125364, 20180103879, 20180098699, 20170311897, 20170215815, 20170191871, 20170191870, 20170049371, 20160345874, 20160242685, 20140170765, 20140018644, assigned to Senseonics, Inc., the entire disclosures of which are hereby incorporated by reference.

However, these sensors are slowly surrounded by an encapsulation tissue that can cause non-accurate measurements that require many invasive calibration tests. In addition, the materials of these sensors need to be replaced every several months, therefore requiring many implantation surgeries for replacing and/or filling and/or recharging the sensors, with the associated, pain, risks, discomfort and potential scars.

There are attempts to prolong the use of the implantable measuring device beyond several months. However, these solutions are aiming for up to one-year use and in many cases requiring multiple invasive calibration tests because of the encapsulation problem.

Some devices have a battery that needs to be replaced or charged. There are also devices in other fields, which are charged by wireless methods like Bluetooth or by Wi-Fi and no replacement is needed. See for example U.S. patent applications No. 20190131825, 20190018472, 20180331865, assigned to Wiliot Ltd. the entire disclosures of which are hereby incorporated by reference.

Several of the patent applications mentioned above describe also an insulin pump that will receive signals from the sensor and deliver insulin accordingly. There were also attempts to develop other types of drug delivery solutions including intra-oral devices for drug delivery and other measurements and treatments. See for example, U.S. Pat. No. 5,298,017, to Theeuwes, et al., U.S. Pat. No. 5,674,196, to Donaldson, et al., U.S. Pat. No. 5,961,482, to Chien, et al., U.S. Pat. No. 5,983,131, to Weaver, et al., U.S. Pat. No. 5,983,134, to Ostrow, and U.S. Pat. No. 6,477,410, to Henley, et al., U.S. Pat. Nos. 6,002,961 and 6,018,678 to Mitragotri, et al., U.S. Pat. Nos. 6,190,315 and 6,041,253 to Kost, et al., U.S. Pat. No. 5,947,921 to Johnson, et al. and U.S. Pat. Nos. 6,491,657, and 6,234,990 to Rowe, et al. U.S. Pat. No. 6,471,696, to Berube, et al., U.S. Pat. No. 6,443,945, to Marchitto, et al., U.S. Pat. No. 4,869,248, to Narula, U.S. Pat. Nos. 6,148,232 and 5,983,135, to Avrahami, U.S. Pat. No. 5,614,223, to Sipos, U.S. Pat. No. 5,686,094, to Acharya, U.S. Pat. No. 6,143,948, to Leitao, et al., U.S. Pat. No. 3,153,855 of Holland, U.S. Pat. No. 3,503,127 to Kasdin and U.S. Pat. No. 4,106,501 to Ozbev, U.S. Pat. No. 4,418,702, to Brown et al., U.S. Pat. No. 5,103,386, to Goldstein et al., U.S. Pat. No. 5,339,829 to Thieme et al., U.S. Pat. No. 5,479,937 to Thieme et al., U.S. Pat. No. 5,563,073 to Titmas, T, U.S. Pat. No. 5,573,009 to Thieme et al., U.S. Pat. Nos. 3,624,909; 3,688,406; 4,020,558; 4,175,326; 4,681,544, 4,685,883, 4,837,030, 4,837,030, 4,919,939, 4,629,424, 6,010,463, 4,321,251, 4,397,944, 5,022,409, 6,143,948, 4,629,424, 4,948,587, 5,458,140, 5,786,227, 6,010,463, 6,152,887, 652,141, 6,212,433, 6,618,627, 6,314,324, 3,732,087, 5,891,185, 6,470,200, 6,430,422, 6,263,223, 5,989,023, 4,629,424, 5,691,539, 6,652,141, 20120220986, 20110076636, 20100312311, 20090210032, 20070106138 and 20040158194, the entire disclosures of which are hereby incorporated by reference.

There are several designs of bone implants, dental implants, hollow dental implants and cannulas for insertion inside the bones and jaws for example as described in U.S. patent application 20130144144 to Laster et al., U.S. patent application 2017049393 A1 to Hyun Ki Bong [KR], U.S. patent application 2004147906 A1 to Voyiazis et al., U.S. patent application 2018035946 A1 to Klab LLC, Chinese patent application CN 208625878 U to Shenzhen Yashang Tech Co Ltd, U.S. patent application no. 20130224687, 20190008615, U.S. Pat. Nos. 8,622,739 and 9,744,057 to Karmon, U.S. Pat. No. 9,271,812 B2 to Richard Cottrell the entire disclosures of which are hereby incorporated by reference.

Therefore, there is a need for an implanted measuring device that can function for several years without additional invasive surgeries and with less repeated calibration tests.

All publications, patents and patent applications referred to above are incorporated by reference in their entirety to the same extent as if each individual publication, patent or patent application was specifically and individually indicated to be incorporated by reference in its entirety (including all references incorporated by reference therein). However, any incorporation by reference of documents above is limited such that no subject matter, definitions, disclaimers, disavowals and inconsistencies are incorporated that is contrary to the explicit disclosure herein, in which case the language in this disclosure controls. Any incorporation by reference of documents above is further limited such that no claims included in the documents are incorporated by reference herein. In other words, any information in any material (e.g., a United States patent, United States patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

### SUMMARY OF THE INVENTION

The present invention provides devices and methods for measuring body parameters and/or agents and/or analytes levels in the human body.

The present invention will demonstrate mainly using a dedicated device which is inserted inside the bones and especially jawbones. The device can be inserted similarly to the insertion of dental implants. The device can be inserted in a location suitable for a dental implant and can also function as a dental implant. The device can be inserted in places in which regular dental implants are not inserted and not to function as a dental implant. The device can be inserted outside the mouth.

A dental implant is a device, which is inserted inside a jawbone and connected to dental component that protrudes to the oral cavity for many years. Therefore, a measuring device can be inserted inside the jawbone and/or inside a unique dental implant, to be there for many years and can be accessible and/or replaced without any surgical procedures. This is because dental implants are made from materials which are not surrounded by encapsulation tissue, like titanium, platinum, palladium, tantalum, molybdenum, zirconium, biocompatible polymers and any combination thereof. Therefore, a measuring sensor in the surrounding of a dental implant can deliver stable and reliable results for a long period of time.

For example, continuous glucose monitoring device like the devices of DexCom Inc., Abbott Inc. and/or Senseonics Inc., Lifecare AS mentioned above, can be inserted inside such a unique dental implant instead inside the skin.

The device can include except for the dental implant, for example, a waterproof sensor/transmitter (Separate or built in), and wireless system between the transmitter and a receiver, for example Bluetooth, wifi, infrared or other means. The sensors can be re-usable and/or replaced. The receiver can be a smartphone and/or other devices that can have for example multiple on-screen trend graphs, direction and rate-of-change arrows. The receiver can be also a smartphone application or the data can be sent to a cloud. The system can warn of falling blood sugar levels. It can send a message to apps on the smartphones of patients or their close contacts.

Other objects and features of the present invention will become apparent in the following detailed description when taken in connection with the accompanying drawings which disclose several embodiments of the invention. It is to be understood that the drawings are designed for the purpose of illustration only and are not intended as a definition of the limits of the invention.

It is also to be understood that any combination of the embodiments described hereafter can be used although these combinations are not explicitly described. The number of possible combinations of different elements in different relations to each other and the number of options of using the devices is enormous. Therefore, only several embodiments and several combinations are described and illustrated, while many combinations can be used although these combinations of features are not described.

Thus, according to the teachings of the present invention there is provided a method for monitoring human body analyte comprising:
a. inserting inside a jaw a dental implant, the dental implant has a coronal part of the dental implant and an apical part of the dental implant, the coronal part has a proximal opening and an internal chamber extending apically from the proximal opening trough the coronal part inside the apical part, the external walls of the apical part has at least one measuring region, which can be at least partially translucent;
b. inserting an analyte measuring component inside the internal chamber;
c. activating a light emitting component to emit light through the measuring region inside the jaw's tissues so the analyte measuring component receives the reflection of the light to measure the analyte.
d. transmitting the measuring result of the analyte measuring component to a receiver.

Thus, according to the teachings of the present invention there is provided a device for monitoring human body analyte comprising:

a dental implant, an analyte measuring component and a light emitting component, the dental implant has a coronal part and an apical part, the coronal part has a proximal opening and an internal chamber extending apically from the proximal opening trough the coronal part inside the apical part, at least part of the analyte measuring component being inside the internal chamber, the external walls of the apical part has at least one measuring region, which can be at least partially translucent, so when the apical part being inside a jaw, the light emitting component emits light through the measuring region inside the jaw's tissues so the analyte measuring component receives the reflection of the light to measure the analyte.

According to a further feature of the present inventions, the at least one measuring region being at a wall of the apical part of the dental implant.

According to a further feature of the present inventions, the at least one measuring region being at an apical end of the apical part of the dental implant.

According to a further feature of the present inventions, the analyte measuring component being connected to the dental implant in a sealed manner to prevent bacteria from entering from the oral cavity inside the internal chamber.

According to a further feature of the present inventions, the coronal part of the dental implant being connected to a protruding component that protrudes to the oral cavity.

According to a further feature of the present inventions, the at least part of the analyte measuring component being inside the protruding component.

According to a further feature of the present inventions, the dental implant and the protruding component are one-piece.

According to a further feature of the present inventions, the dental implant and the protruding component are connected in a detachable connection.

According to a further feature of the present inventions, the protruding component being wider than the dental implant.

According to a further feature of the present inventions, the protruding component protrudes buccally from the dental implant to be adjacent the cheek.

According to a further feature of the present inventions, the protruding component protrudes buccally from the dental implant to be at the buccal vestibulum.

According to a further feature of the present inventions, the protruding component function as a dental prosthesis selected from the group consisting of: a crown, a bridge, a denture, an abutment, a supra-structure, an infra-structure and any combination thereof.

According to a further feature of the present inventions, the protruding component being connected to the dental implant in a sealed manner to prevent bacteria from entering from the oral cavity inside the internal chamber.

According to a further feature of the present inventions, the protruding component being at least partially translucent to enable light from the oral cavity to enter the internal chamber.

According to a further feature of the present inventions, the protruding component has at least one undercut for fixation of a rubber dam.

According to a further feature of the present inventions, the protruding component includes an energy source.

According to a further feature of the present inventions, the energy source being charged by movements of the jaw in which the dental implant being inserted.

According to a further feature of the present inventions, the energy source being charged by chewing on the protruding component and/or by the tongue pushing the protruding component.

According to a further feature of the present inventions, the protruding component includes a transducer that transduce a measuring result of the analyte measuring component to a receiving component, said transducer being activated by chewing on said protruding component and/or by the tongue pushing the protruding component.

According to a further feature of the present inventions, a movement of the protruding component caused by chewing over the protruding component and/or by the tongue pushing the protruding component activates the analyte measuring component to measure the analyte.

According to a further feature of the present inventions, at least part of the light emitting component being located inside the protruding component.

According to a further feature of the present inventions, at least part of the light emitting component being located inside the dental implant.

According to a further feature of the present inventions, the analyte measuring component being connected to the dental implant by a flexible connector to enable movements of the analyte measuring component inside the internal chamber during mastication.

According to a further feature of the present inventions, an external surface of the apical part of the dental implant adjacent the at least one measuring region has smooth external surface region to prevent attachment of bone tissue to the smooth external surface region.

According to a further feature of the present inventions, the apical part of the dental implant has at least one rough external surface region to promote bone tissue attachment to the rough external surface region and at least one smooth external surface region to prevent attachment of bone tissue to the smooth external surface region.

According to a further feature of the present inventions, the smooth external surface region being adjacent the at least one measuring region.

According to a further feature of the present inventions, the apical part of the dental implant has at least one rough external surface region to promote bone tissue attachment to the rough external surface region and at least one less rough external surface region to reduce attachment of bone tissue to the less rough external surface region compared to the attachment of bone to the rough external surface.

According to a further feature of the present inventions, the less rough external surface region being adjacent the at least one analyte permeable region.

According to a further feature of the present inventions, an external surface of the apical part includes a material that promotes angiogenesis.

According to a further feature of the present inventions, the coronal part of the dental implant includes a connection with an anti-rotational element for receiving a dental component with a matching anti-rotational element.

According to a further feature of the present inventions, the dental implant has a core and at least one external thread extending along at least part of the core.

According to a further feature of the present inventions, the analyte selected from the group consisting of: glucose, cholesterol, lipids, iron, ferritin, Natrium, potassium, salts, immunoglobulins, oxygen saturation, liver enzymes, hormones and any combination thereof.

According to a further feature of the present inventions, part of the dental implant being part of the analyte measuring component.

According to a further feature of the present inventions, the light emitting component emits light from the oral cavity.

Thus, according to the teachings of the present inventions, there is provided a method for monitoring human body analyte comprising:
a. Inserting inside a jaw a dental implant, the dental implant has a coronal part of the dental implant and an apical part of the dental implant, the apical part of the dental implant has an internal chamber having at least one analyte permeable region configured to allow entrance of interstitial fluids and the analyte from the jaw inside the internal chamber through the at least one analyte permeable region while preventing entrance of cells from the jaw inside the internal chamber through the at least one analyte permeable region;
b. inserting an analyte measuring component inside the dental implant to measure the analyte inside the internal chamber;
c. transmitting the measuring result of the analyte measuring component to a receiver.

Thus, according to the teachings of the present inventions, there is provided a dental implant for monitoring human body analyte comprising:

a coronal part of the dental implant and an apical part of the dental implant, the apical part of the dental implant has an internal chamber and at least one distal opening configured so when the apical part of the dental implant being inside a jaw, interstitial fluids and the analyte can enter from the jaw inside the internal chamber through the at least one distal opening, the apical part of the dental implant has an analyte permeable region configured so when the apical part of the dental implant being inside a jaw, interstitial fluids and the analyte can enter from the jaw inside the internal chamber through the at least one analyte permeable region while preventing cells to enter from the jaw inside the internal chamber through the at least one analyte permeable region to enable an analyte measuring component to measure the analyte inside the internal chamber.

Thus, according to the teachings of the present inventions, there is provided a device for monitoring human body analyte comprising:

a dental implant and an analyte measuring component, the dental implant has a coronal part and an apical part, the dental implant has an internal chamber extending from a proximal opening in the coronal part inside the apical part, the apical part has at least one analyte permeable region, so when the apical part being inside a jaw, interstitial fluids and the analyte can enter from the jaw inside the internal chamber through the at least one analyte permeable region to enable the analyte measuring component to measure the analyte, while cells are prevented from entering the internal channel.

Thus, according to the teachings of the present inventions, there is provided a dental implant for monitoring human body analyte comprising:

a coronal part of the dental implant and an apical part of the dental implant, the coronal part of the dental implant has a proximal opening, the apical part of the dental implant has at least one analyte permeable region allowing, when the apical part of the dental implant being inside a jaw, the entrance of interstitial fluids and the analyte from the jaw through the analyte permeable region inside the apical part of the dental implant while preventing cells to enter from the jaw through the analyte permeable region inside the apical part of the dental implant to form an internal chamber inside the apical part filled with the interstitial fluids and the analyte without cells to enable an analyte measuring component inserted inside the dental implant through the proximal opening of the coronal part of the dental implant to measure the analyte inside the internal chamber.

Thus, according to the teachings of the present inventions, there is provided a dental implant for monitoring human body analyte comprising:

a coronal part of the dental implant and an apical part of the dental implant, the apical part of the dental implant has an internal chamber and at least one analyte permeable region configured so when the apical part of the dental implant being inside a jaw, interstitial fluids and the analyte can enter from the jaw inside the internal chamber through the at least one analyte permeable region while preventing cells to enter from the jaw inside the internal chamber through the at least one analyte permeable region to enable an analyte measuring component to measure the analyte inside the internal chamber.

Thus, according to the teachings of the present inventions, there is provided a jawbone implant for monitoring human body analyte comprising:

a coronal part of the jawbone implant and an apical part of the jawbone implant, the apical part of the jawbone implant has an internal chamber and a capillaries chamber, the capillaries chamber has at least one perforated wall having at least one perforation of a diameter of at least 5 micrometers that enables entrance of capillaries from a jawbone inside the capillaries chamber when the apical part of the jawbone being inside a jawbone, the jawbone implant has further at least one analyte permeable region between the capillaries chamber and the internal chamber configured so when the apical part of the jawbone implant being inside the jawbone, interstitial fluids and the analyte can enter from the capillaries chamber inside the internal chamber through the at least one analyte permeable region, a largest diameter of a perforation in the analyte permeable region being smaller than 1 micrometer to prevent cells to enter from the capillaries chamber inside the internal chamber through the at least one analyte permeable region, the internal chamber being accessible from the coronal part of the jawbone implant to enable insertion of an analyte measuring component inside the internal chamber through the coronal part of the jawbone implant to measure the analyte inside the internal chamber.

According to a further feature of the present inventions, the analyte permeable region being perforated, a largest diameter of a perforation in the analyte permeable region being smaller than 1 micrometer.

According to a further feature of the present inventions, the analyte permeable region being perforated, a largest diameter of a perforation in the analyte permeable region being smaller than 0.3 micrometer.

According to a further feature of the present inventions, the analyte permeable region being perforated, a largest diameter of a perforation in the analyte permeable region being smaller than 0.1 micrometer.

According to a further feature of the present inventions, the analyte permeable region being perforated, a largest diameter of a perforation in the analyte permeable region being smaller than 50 nanometer.

According to a further feature of the present inventions, the analyte permeable region being perforated, a largest diameter of a perforation in the analyte permeable region being smaller than 10 nanometer.

According to a further feature of the present inventions the dental implant has a capillaries chamber, the capillaries chamber has at least one perforated wall that enables entrance of capillaries from the jaw inside the capillaries chamber while preventing the formation of dense bone tissue inside the capillaries chamber, the capillaries chamber being separated from the internal chamber of the dental implant by the analyte permeable region.

According to a further feature of the present inventions the perforated wall of the capillaries chamber that enables entrance of capillaries has several pores having a diameter of 30-2000 micrometers.

According to a further feature of the present inventions the perforated wall of the capillaries chamber that enables entrance of capillaries has several pores having a diameter of more than 2000 micrometers.

According to a further feature of the present inventions the perforated wall of the capillaries chamber that enables entrance of capillaries has several pores having a diameter of more than 100-500 micrometers.

According to a further feature of the present inventions the perforated wall of the capillaries chamber that enables entrance of capillaries has several pores having a diameter of more than 100 micrometers.

According to a further feature of the present inventions the capillaries chamber has at least two separate regions with pores of 30-80 micrometers

According to a further feature of the present inventions the capillaries chamber has rough internal surface.

According to a further feature of the present inventions the capillaries chamber has a material that promotes angiogenesis.

According to a further feature of the present inventions, the dental implant further includes a semi-permeable membrane that allows the passage of the analyte from the jaw through the semi-permeable membrane inside the internal chamber while preventing the passage of molecules which are 5 times larger than the analyte.

According to a further feature of the present inventions, the analyte permeable region prevents the passage of bacteria from the internal chamber to the surrounding tissue through the analyte permeable region.

According to a further feature of the present inventions, the dental implant being part of a device that includes the analyte measuring component.

According to a further feature of the present inventions, the at least one analyte permeable region being at a wall of the apical part of the dental implant.

According to a further feature of the present inventions, the at least one analyte permeable region being inside the apical part of the dental implant.

According to a further feature of the present inventions, the at least one analyte permeable region being part of a semi-permeable barrier located inside the apical part of the dental implant.

According to a further feature of the present inventions, wherein the apical part of the dental implant being perforated to enable entrance of the interstitial fluids and the analyte from the jaw inside the apical part of the dental implant, an envelope being inside the apical part of the dental implant, the at least one analyte permeable region being part of the envelope so the internal chamber being at least partially surrounded by the envelope to prevent entrance of cells inside the internal chamber.

According to a further feature of the present inventions, wherein the envelop has a tube shape.

According to a further feature of the present inventions, the envelop has a bag shape.

According to a further feature of the present inventions, the apical part of the dental implant being perforated to enable entrance of the interstitial fluids and the analyte from the jaw inside the apical part of the dental implant, an envelope being inside the apical part of the dental implant, the at least one analyte permeable region being part of the envelope so the internal chamber being at least partially surrounded by the envelope to prevent entrance of cells inside the internal chamber.

According to a further feature of the present inventions, wherein the envelop being fixated to the internal walls of the dental implant by at least one screw.

According to a further feature of the present inventions, an apical region of the envelop being fixated to the internal walls of the dental implant by an apical fixating screw, a coronal region of the envelop being fixated to the internal walls of the dental implant by a hollow fixating screw.

According to a further feature of the present inventions, a wall of the internal chamber has at least one distal opening that allows entrance of the interstitial fluids and the analyte inside the internal chamber, the at least one distal opening being covered by a semi-permeable membrane that allows passage of the interstitial fluids and the analyte from the jaw inside the internal chamber while preventing passage of cells from the jaw inside the internal chamber.

According to a further feature of the present inventions, the distal opening being part of a mesh.

According to a further feature of the present inventions, a wall of the internal chamber being a wall of the apical part of the dental implant.

According to a further feature of the present inventions, a wall of the internal chamber being a wall of an envelope inside the apical part of the dental implant.

According to a further feature of the present inventions, the envelope being screwed inside the dental implant.

According to a further feature of the present inventions, a majority of the walls of the internal chamber are the internal walls of the dental implant.

According to a further feature of the present inventions, a majority of the walls of the internal chamber are a semi-permeable barrier located inside the dental implant.

According to a further feature of the present inventions, the analyte measuring component being covered by a covering membrane, the analyte permeable region allows the passage of larger molecules than the molecules that the covering membrane allows.

According to a further feature of the present inventions, the coronal part of dental implant has a proximal opening, the analyte measuring component being at least partially inserted inside the internal chamber through the proximal opening of the coronal part of the dental implant.

According to a further feature of the present inventions, the analyte measuring component being connected to the dental implant in a sealed manner to prevent bacteria from entering from the oral cavity inside the internal chamber.

According to a further feature of the present inventions, at least part of the analyte measuring component being inside the internal chamber.

According to a further feature of the present inventions, the coronal part of the dental implant being connected to a protruding component that protrudes to the oral cavity.

According to a further feature of the present inventions, the at least part of the analyte measuring component being inside the protruding component.

According to a further feature of the present inventions, the dental implant and the protruding component are one-piece.

According to a further feature of the present inventions, the dental implant and the protruding component are connected in a detachable connection.

According to a further feature of the present inventions, a semi-permeable membrane can be located between the dental implant and the protruding component.

According to a further feature of the present inventions, the protruding component being wider than the dental implant.

According to a further feature of the present inventions, the protruding component protrudes buccally from the dental implant to be adjacent the cheek.

According to a further feature of the present inventions, the protruding component protrudes buccally from the dental implant to be at the buccal vestibulum.

According to a further feature of the present inventions, the protruding component function as a dental prosthesis selected from the group consisting of: a crown, a bridge, a denture, an abutment, a supra-structure, an infra-structure and any combination thereof.

According to a further feature of the present inventions, the protruding component being connected to the dental implant in a sealed manner to prevent bacteria from entering from the oral cavity inside the internal chamber.

According to a further feature of the present inventions, the protruding component being at least partially translucent to enable light from the oral cavity to enter the internal chamber.

According to a further feature of the present inventions, the protruding component has at least one undercut for fixation of a rubber dam.

According to a further feature of the present inventions, the protruding component includes an energy source.

According to a further feature of the present inventions, the energy source being charged by movements of the jaw in which the dental implant being inserted.

According to a further feature of the present inventions, the energy source being charged by chewing on the protruding component and/or by the tongue pushing the protruding component.

According to a further feature of the present inventions, the protruding component includes a transducer that transduce a measuring result of the analyte measuring component to a receiving component, said transducer being activated by chewing on the protruding component and/or by the tongue pushing the protruding component.

According to a further feature of the present inventions, a movement of the protruding component caused by chewing over the protruding component and/or by the tongue pushing the protruding component activates the analyte measuring component to measure the analyte.

According to a further feature of the present inventions, at least part of a light emitting component being located inside the protruding component.

According to a further feature of the present inventions, at least part of a light emitting component being located inside the internal chamber.

According to a further feature of the present inventions, the analyte measuring component further includes a light emitting component which emits light inside the internal chamber.

According to a further feature of the present inventions, the dental implant being at least partially translucent to enable light from the oral cavity to enter the internal chamber.

According to a further feature of the present inventions, a circulating element being inside the dental implant to move fluids inside the internal chamber.

According to a further feature of the present inventions, the analyte measuring component being connected to the dental implant by a flexible connector to enable movements of the analyte measuring component inside the internal chamber during mastication.

According to a further feature of the present inventions, an external surface of the apical part of the dental implant adjacent the at least one analyte permeable region has smooth external surface region to prevent attachment of bone tissue to the smooth external surface region.

According to a further feature of the present inventions, the apical part of the dental implant has at least one rough external surface region to promote bone tissue attachment to the rough external surface region and at least one smooth external surface region to prevent attachment of bone tissue to the smooth external surface region.

According to a further feature of the present inventions, the smooth external surface region being adjacent the at least one analyte permeable region.

According to a further feature of the present inventions, the apical part of the dental implant has at least one rough external surface region to promote bone tissue attachment to the rough external surface region and at least one less rough external surface region to reduce attachment of bone tissue to the less rough external surface region compared to the attachment of bone to the rough external surface.

According to a further feature of the present inventions, the less rough external surface region being adjacent the at least one analyte permeable region.

According to a further feature of the present inventions, an external surface of the apical part includes a material that promotes angiogenesis.

According to a further feature of the present inventions, the internal chamber includes a material selected from the group consisting of bacteriostatic material, bacteriocidic material, antibiotics and any combination thereof.

According to a further feature of the present inventions, the internal chamber includes a measuring solution forming a chemical reaction with the analyte while the analyte measuring component being configured to measure the chemical reaction.

According to a further feature of the present inventions, the measuring solution can be replaced trough a proximal opening in the coronal part of the dental implant while preventing bacteria from the oral cavity to enter the internal chamber.

According to a further feature of the present inventions, the internal chamber has bacteria producing at least part of the measuring solution.

According to a further feature of the present inventions, the coronal part of the dental implant includes a connection with an anti-rotational element for receiving a dental component with a matching anti-rotational element.

According to a further feature of the present inventions, the at least one analyte permeable region being located at a distal end of the apical part of the dental implant.

According to a further feature of the present inventions, the dental implant has a core and at least one external thread extending along at least part of the core.

According to a further feature of the present inventions, the analyte selected from the group consisting of: glucose, cholesterol, lipids, iron, ferritin, Natrium, potassium, salts, immunoglobulins, oxygen saturation, liver enzymes, hormones and any combination thereof.

According to a further feature of the present inventions, part of the dental implant being part of the analyte measuring component.

According to a further feature of the present inventions, the distal opening of the dental implant and the analyte permeable region being located at the distal opening of the dental implant.

According to a further feature of the present inventions, the semi-permeable membrane being along the at least one analyte permeable region.

### BRIEF DESCRIPTION OF THE DRAWINGS

The invention is herein described, by way of example only, with reference to the accompanying drawings, wherein:

FIG. 1 illustrates an elevation view of an embodiment of a dental implant having an opening at its apical part to enable the entrance of interstitial fluids and the analyte inside the apical part of the dental implant.

FIG. 2 illustrates a sectional view of an embodiment of a dental implant having an opening at its apical part to enable the entrance of interstitial fluids and the analyte inside the apical part of the dental implant.

FIG. 3 illustrates a sectional view of an embodiment of the dental implant connected by a screwed connection to the analyte measuring component having a sensor inside the internal chamber of the apical part of the dental implant and a protruding component that protrudes to the oral cavity having a transmitter and an energy source.

FIG. 4 illustrated a sectional view of an embodiment of a dental implant with a sensor of an analyte measuring component inside the dental implant and the protruding component with the transmitter, which protrudes from the dental implant to the oral cavity, can be a dental prosthesis.

FIG. 5 illustrates a sectional view of an embodiment of the dental implant connected in a sealed connection to the analyte measuring component having a sensor inside the internal chamber of the apical part of the dental implant and a protruding component that protrudes to the oral cavity having a transmitter and an energy source.

FIG. 6 illustrates a sectional view of an embodiment in which the analyte measuring component can move to refresh the interstitial fluids inside the internal chamber of the dental implant.

FIG. 7 illustrates a sectional view of an embodiment in which the apical part of the dental implant has a distal opening and/or a perforated region being covered by a semi-permeable barrier that can be permeable to the analyte and restrict passage therethrough of substances, e.g., cells and large molecules.

FIG. 8A illustrates a sectional view of an embodiment in which the dental implant has a distal opening and/or a perforated region and the dental implant has inside a tube shape semi-permeable barrier that surrounds the internal chamber of the dental implant and function as the analyte permeable region.

FIG. 8B illustrates a sectional view of an embodiment in which the dental implant has a distal opening and/or a perforated region and the dental implant has inside bag shape semi-permeable barrier that surrounds the internal chamber of the dental implant and has a region that function as the analyte permeable region.

FIG. 9 illustrates a sectional view of an embodiment in which the dental implant has distal opening/s region and the dental implant has inside a tube and/or bag shape semi-permeable barrier that surrounds the internal chamber of the dental implant and function as the analyte permeable region. The analyte measuring component being connected to the dental implant, so the sensor can be inside the bag shape semi-permeable barrier.

FIG. 10A illustrates a sectional view of an embodiment in which the dental implant has distal opening/s region/s and the dental implant has inside a tube and/or bag shape semi-permeable barrier that surrounds the internal chamber of the dental implant and function as the analyte permeable region. The device can allow the movements of the analyte measuring component inside the tube/bag shape semi-permeable barrier. The dental implant can have a chamber for blood vessels like capillaries.

FIG. 10B illustrates a sectional view of an embodiment in which the dental implant has distal opening/s region/s and the dental implant has inside a tube and/or bag shape semi-permeable barrier that surrounds the internal chamber of the dental implant and function as the analyte permeable region. The device can allow the movements of the analyte measuring component inside the tube/bag shape semi-permeable barrier. The dental implant can have a chamber for blood vessels like capillaries.

FIG. 10C illustrates a sectional view of an embodiment in which the dental implant has distal opening/s region/s and the dental implant has inside a tube and/or bag shape semi-permeable barrier that surrounds the internal chamber of the dental implant and function as the analyte permeable region. The device can allow the movements of the analyte measuring component inside the tube/bag shape semi-permeable barrier. The dental implant can have a chamber for blood vessels like capillaries.

FIG. 10D illustrates a sectional view of an embodiment in which the dental implant has opening/s at the apical part that enable the entrance of small blood vessels and the dental implant has inside a tube and/or bag shape semi-permeable barrier that surrounds the internal chamber of the dental implant and function as the analyte permeable region. The device allows the movements of the analyte measuring component inside the tube/bag shape semi-permeable barrier. The dental implant can have a chamber for blood vessels like capillaries.

FIG. 11A illustrates a perspective view of an embodiment of a capsule having the analyte measuring component for insertion inside the dental implant.

FIG. 11B illustrates a perspective view an embodiment of a capsule having the analyte measuring component for insertion inside the dental implant.

FIG. 12 illustrates a sectional view of an embodiment of a medicine/insulin pump activated by a piston as part of a dental implant and a dental prosthesis like a dental crown.

FIG. 13 illustrates a sectional view of an embodiment of a peristaltic medicine pump as part of a dental implant and a dental prosthesis like a dental crown.

FIG. 14 illustrates a sectional view of an embodiment of a dental implant having a peristaltic medicine pump, an analyte measuring component and a dental prosthesis.

FIG. 15 illustrates a block/flow diagram of an embodiment of the device that include the dental implant, sensors, insulin pump, transmitters, receivers, memory storage device and alarm system.

FIG. 16 illustrated a sectional view of an embodiment of a jawbone implant inside the anterior mandible below a central incisor.

### DESCRIPTION OF THE PREFERRED EMBODIMENTS

Before turning to the features of the present invention in more detail, it will be useful to clarify certain terminology as will be used herein in the description and claims.

The device can be used to measure many parameters. The body parameters can be biological agents and/or analytes like glucose, cholesterol, lipids, iron, ferritin, Natrium, potassium, salts, immunoglobulins, oxygen saturation, liver enzymes, hormones etc. The body parameters can be for example body temperature, tissue and/or blood pressure, E.C.G., vascular resistance, heart rate, cardiac output, gases in breading, respiration rate, apneas, snoring, body and head movement and more.

Parameters can be also geographic location and/or height, atmospheric pressure, velocity, acceleration, tremor, biting force etc. In the present patent application, the terms “parameter”, “analyte”, “agent” will be used interchangeably. Accordingly, and for conciseness of presentation, only one of these terms will generally be used in the following description, without implying the exclusion of the other classes of materials and parameters. The description will also mention glucose as an example, while other analytes are also possible.

The device can transmit the data in real time and/or collect the data, for example during the night for transmitting the next day, for example via the cloud, to the patient himself and/or patient's family and/or patient's doctor for monitoring. etc.

The use of the device will be mainly described for the jawbone, however, other bones in the skull and in the body can be used, for example the, zygoma bone. Therefore, the terms “dental implant”, “jawbone implant”, “bone implant”, “skull implant” or “mouth implant” are used interchangeably to describe an element that can be fixated inside a bone. Accordingly, and for conciseness of presentation, only one of these terms will generally be used in the following description, without implying the exclusion of the other. The bone can be in the skull preferably the mandible and maxilla allowing the access to the bone implant from the oral cavity and the implant can protrude to the oral cavity and/or to be connected to elements that protrude to the oral cavity for several years. The implant can be also protruding outside the skin, for example near the ear.

Some features of components can have at least two options and are described as “xx/xy” or “xx and/or xy”. If only one such feature is mentioned later it still can be, if applicable, also the second feature and similar features.

The dental implant and/or jawbone implant and/or bone implant can have an internal “channel” or internal “chamber”. Both terms are used interchangeably to describe a space inside the dental implant that can be occupied by various elements and materials and surrounded by various elements and materials Accordingly, and for conciseness of presentation, only one of these terms will generally be used in the following description, without implying the exclusion of the other. The internal channel and/or internal chamber can have materials coming from the tissue and/or materials inserted by the patient and/or medical personnel and/or have components of the device. The surrounding of the internal chamber can be the walls of the dental implant and/or an envelope inserted inside the dental implant, while this envelope being surrounded by the walls of the dental implant.

Finally, with respect to terminology, the term “distal end” or “distal part” means the side of an element that is closer to the patient. The term “proximal end” or “proximal part” means the side of the element that is close to the physician. “Distally” means more towards the patient and “proximally” closer to the physician.

Turning now in detail to the drawings, which depict several embodiments of the invention for the purpose of illustrating the practice thereof and not by way of limitation of the scope of the invention, and in which reference characters refer to corresponding elements throughout the several views.

It should be noted that in some drawings parts of the device are not touching each other although these parts can touch or in some cases should touch each other. The separation/s between the parts are for better discriminating between the parts that in some cases can be difficult when these parts are touching each other. For example, when a thin semi-permeable membrane is lining the inner walls of the dental implant and touching these inner walls, it might be difficult to see the membrane.

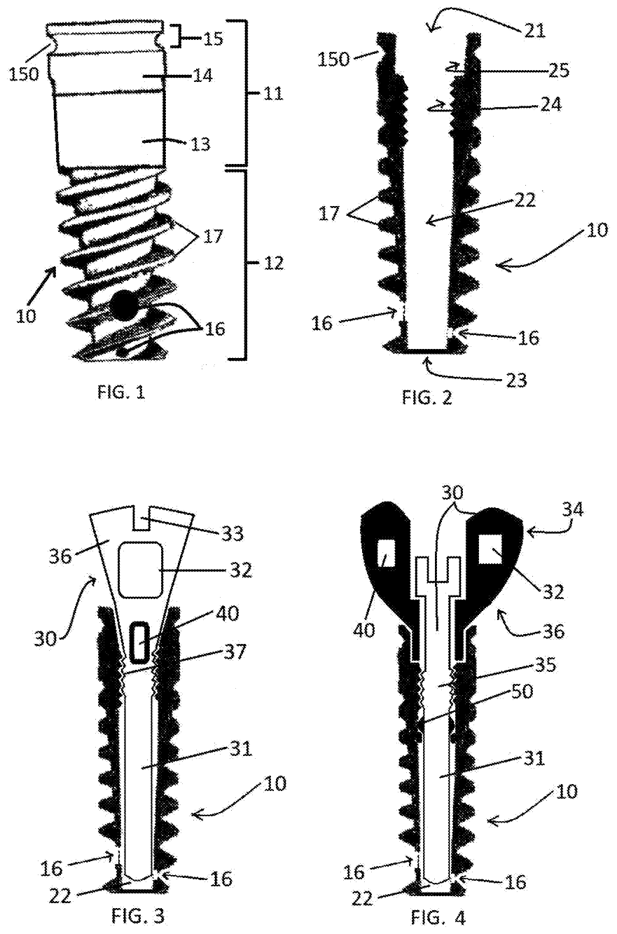

The new device includes a bone implant like a dental implant and an analyte measuring component. FIG. 1 illustrates an elevation view of an embodiment of such a dental implant 10 and FIG. 2 illustrates a sectional view of an embodiment of such a dental implant 10. The dental implant 10 can have a coronal part 11 and an intra-bony apical part 12. The coronal part 11 can have three regions: an intra-bony region 13 to be inside the bone, an intra-gingival region 14 to be inside the soft tissue or the gums and a supra-gingival region 15 to protrude to the oral cavity. In other embodiments the entire coronal part 11 can be inside the bone or the entire coronal 11 part can be inside the gums or the entire coronal part 11 can be above the gums protruding to the oral cavity. The coronal part 11 can have a proximal opening 21 leading to an internal chamber 22 illustrated in FIG. 2. The apical part 12 can have an analyte permeable region/s 16 that will enable interstitial fluids and the analyte to pass through the analyte permeable region 16 from the surrounding tissues to enter inside the internal chamber 22 of the bone implant 10, while preventing the entrance of cells and optionally also molecules larger than the analyte from the surrounding tissues inside the internal chamber 22. The analyte permeable region 16 can be at a side wall of the apical part 12 of the dental implant 10 as illustrated in FIGS. 1 and 2 and/or can be at the apical end 23 of the dental implant 10 and/or other locations. The apical part 12 of the dental implant 10 can have several analyte permeable regions 16. An analyte permeable region can be also at the coronal part 11 of the dental implant 10. The apical part 12 can have for example at least one region with many small perforations to serve as the analyte permeable region 16. The proximal opening 21 and the analyte permeable region 16 can be connected by an internal channel and/or internal chamber 22 as illustrated in FIG. 2, so after insertion of the dental implant 10 inside the jawbone, materials from the jawbone, including interstitial fluids and the analyte, can enter the internal chamber 22. The analyte permeable region can be for example a hole or have several holes with a diameter larger than 1 mm that will allow the entrance of tissue and blood vessels. The analyte permeable region can be for example a hole or several holes with a diameter smaller than 1 mm and/or smaller than 0.1 mm and/or smaller than 30 micrometers and/or smaller than 10 micrometers and/or smaller than 5 micrometers and/or smaller than 1 micrometer and/or smaller than 0.3 micrometer and/or smaller than 0.1 micrometer and/or smaller than 50 nanometer and/or smaller than 30 nanometer and/or smaller than 10 nanometer that will allow the entrance of interstitial fluids and some analytes while blocking the entrance of cells and blood vessels. The analyte permeable region can have for example a hole or several holes with a diameter larger than 1 mm and a hole or several holes with a diameter smaller than 1 mm. The jawbone implant can have more than one analyte permeable region, so a first analyte permeable region has hole/s with a first range of diameters and a second analyte permeable region has hole/s with a second range of diameters, while the second range being smaller than the first range. The jawbone implant can have more than one internal channel. For example, the first analyte permeable region can lead to a first internal channel and the second analyte permeable channel can lead to a second internal channel. The dental implant 10 can have an internal thread 24 inside the internal chamber 22 and/or coronally to internal chamber 22 to enable connection to a dental abutment. The jawbone implant 10 can have an anti-rotational element 25 that can be internal and/or external for the insertion of the jawbone implant 10 by rotating the jawbone implant like a conventional dental implant having an external thread. The jawbone implants 10 of FIGS. 1 and 2 illustrate embodiments of a dental implant with two external threads 17. The thread/s can be along at least part of the apical part 12 and/or along at least part of the coronal part 11 of the dental implant.

The analyte measuring component can be a sensor and/or a sensor+transmitter and/or sensor+transmitter+receiver. In most of the embodiments, the sensor and transmitter are inside the mouth while the receiver can be outside the mouth. However, in some embodiments, the receiver can be inserted inside the mouth for receiving the signal from the transmitter and/or sensor and then taken out. The receiver can be also part of an insulin/medicine pump that can be inside the mouth. The analyte measuring component can be at least partially or completely inside the internal chamber 22 of the dental implant 10 or can be at least partially outside the internal chamber 22.

The sensor can also measure for example the pressure inside the internal chamber of the dental implant, which can reflect the blood pressure.

In addition to the dental implant 10 and the analyte measuring component, the device further can include a protruding component that protrudes from the dental implant 10 in the jawbone to the oral cavity. The protruding component can be one-piece with the dental implant 10 or can be a separate component which can be connected to the dental implant. The analyte measuring component can be at least partially or completely inside the protruding component.

FIG. 3 illustrates an embodiment in which the analyte measuring component 30 has a sensor 31, which can be at least partially inside the internal chamber 22 of the dental implant 10 surrounded by the interstitial fluids and the analyte while the transmitter 32 can be part of the protruding component 36 being at least partially outside the dental implant 10 protruding to the oral cavity.

The sensor 31 and transmitter 32 can be one-piece so to be inserted and removed together as illustrated in FIG. 3, in which the analyte measuring component 30 has an external thread 37 that matches the internal thread 24 of the dental implant 10, so the analyte measuring component 30 can be fixated by screwing to the dental implant 10. The analyte measuring component 30 can have a socket with an anti-rotational shape 33, for example, internal polygon like hexagonal shape and/or square and/or octagon to enable screwing the analyte measuring component 30 to the dental implant 10. The sensor 31 and transmitter 32 can be detachable to enable replacing only the transmitter or only the sensor. The protruding component 36 and/or the sensor 31 can include an energy source 40 like a small battery.

FIG. 4 illustrated an embodiment in which the protruding component 36 can be a dental prosthesis 34, for example, a dental crown and/or abutment. The transmitter 32 can be part of the dental prosthesis 34 and the sensor 31 can be part of a screw 35 that fixates the transmitter 32 and the dental crown 34 to the dental implant 10.

In the embodiment of FIG. 4, if the dental crown 34 is fixated to the dental implant 10 also by friction, for example, by having a conical connection or Morse connection or fixated using a resilient band or other means except for the screw 35, then the sensor 31 can be replaced every several weeks or months while the transmitter 32 remains in place with the dental crown 34.

The analyte measuring component can be the screw 35 or the analyte measuring component 30 can be the combination of the screw 35 with the dental crown 34, as illustrated in in FIG. 4. The sensor 31 of the analyte measuring component 30 can be at least partially inside the internal chamber 22 of the dental implant 10 while the transmitter 32 of the analyte measuring component 30 can be at least partially inside the protruding component 36 and/or dental prosthesis 34. The protruding component 36 and/or the sensor 31 can include an energy source 40 like a small battery.

The dimensions of the jawbone implant 10 and the internal channel 22 can be limited by the dimensions of the jawbone, while the dimensions of the protruding component can be limited by the dimensions and structures of the oral cavity (teeth, tongue, cheek etc.). Therefore, in several embodiments the diameter of the protruding component 36 will be larger than the diameter of the dental implant 10. Therefore, in several embodiments, part of the analyte measuring component 30 can be part of the protruding component 36.

The dimensions of the elements of the device can vary according to the location of the dental implant 10, the dimensions of the jaw and/or the dimensions of the analyte measuring component 30 and/or transmitter 32. For example, the external diameter of the dental implant 10 in a cross-section, which is perpendicular to the longitudinal axis of the dental implant 10, can be 2-8 mm and/or 3-7 and/or 4-6 mm. For example, the external diameter of the dental implant 10 can be constant or have a variable diameter. For example, the external diameter can be at least partially reduced apically and/or at least partially reduced coronally. For example, the diameter of the internal channel and/or internal chamber, in a cross-section, which is perpendicular to the longitudinal axis of the dental implant 10 can be 0.1-3 mm and/or 0.4-2 mm and/or 0.5-1.5 and and/or 0.7-1.3 mm less than the external diameter of dental implant 10. For example, the diameter of the internal chamber/s can be 0.1-6 mm and/or 0.5-5 mm and/or 2.5-4 mm.

If the dental implant is intended only for holding an analyte measuring component like a continuous glucose monitoring device, then the dental implant can be narrower. Several continuous glucose monitoring devices in the market have a narrow needle for insertion inside the skin having a diameter of less than 1 mm and even less than 0.5 mm. Such a dental implant can have, for example, an external diameter of 1-2 mm and/or internal chamber with a diameter of 0.1-1.5 mm and/or wall thickness of 0.2-0.5 mm.

The apical-coronal length of the dental implant 10 can be, for example, 5-20 mm and/or 6-18 and/or 8-13 mm. The apical-coronal length of the protruding component 36 can be, for example, 3-15 mm and/or 4-12 and/or 5-10 mm. The diameter in a cross-section, which is perpendicular to the longitudinal axis of the dental implant, of the protruding component 36 can be, for example, 3-12 mm and/or 5-10 mm and/or 6-8 mm.

The protruding component 36 can be connected to the dental implant 10 in a variety of options. The dental implant 10 can have internal and/or external thread and the protruding component 36 can have a matching external and/or internal thread so to be screwed to the dental implant 10. The dental implant can have an internal conical surface and the protruding component can have a matching external conical surface, so the protruding component can be inserted inside the dental implant by pushing and being fixated by friction. The connection can be by a click mechanism using a flexible element at the dental implant and/or at the protruding component. The connection between the dental implant and the protruding component can be a sealed connection to prevent bacteria from entering inside the internal chamber 22. For example, the connection between the dental implant and the protruding component can include a flexible ring and/or a resilient band like a rubber and/or silicone ring.

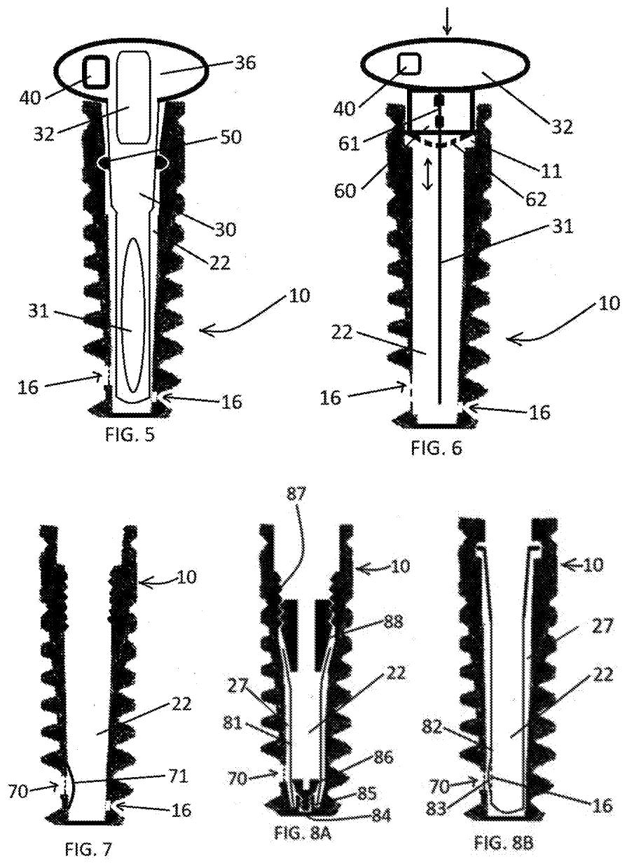

In another embodiment, the analyte measuring component 30 can be inserted at least partially inside the internal chamber 22 and seals at least part of the internal channel 22. For example, the analyte measuring component 30 can include a flexible ring and/or a resilient band like a rubber and/or silicone ring 50 to be in contact with the walls of the internal chamber 22 as illustrated in FIG. 4 or to be inside a slot in the walls of the internal chamber as illustrated in FIG. 5. The protruding component 36 can be placed over the analyte measuring component 30 to protect it. The protruding component 36 can be connected to the dental implant 10 and/or connected to the analyte measuring component 30 in a connection that can be a sealed connection.

FIG. 6 illustrates an embodiment in which the coronal part 11 of the dental implant 10 can be closed and/or sealed by a resilient cover and/or plug 60 made, for example, from silicone, neoprene, latex and/or similar materials. The sensor 31 of the analyte measuring component 30 can be in the shape of a needle (resembling the sensors of the continues glucose monitoring devices in the market like DexCom G6 from DexCom Inc., and/or FreeStyle Libre from Abbott Inc. and/or Guardian sensor 3 from Medtronic Inc.). The sensor 31 can be inserted inside the internal chamber 22 of the dental implant 10 through the resilient cover 60 of the dental implant 10, while the transmitter 32 remains protruding to the oral cavity like the transmitters of the continues glucose monitoring devices in the market, which are protruding on the skin. The resilient cover and/or plug 60 of the dental implant 10 can be part of the sensor 31 and inserted inside the dental implant 10 together with the sensor 31. The sensor can have a region of reduced diameter 61 for fixation in the resilient cover and/or plug 60 as illustrated in FIG. 6. The resilient cover 60 and/or plug can allow some movements of the sensor 31 inside the internal chamber 22, mainly movements along the longitudinal axis of the dental implant. These movements of the sensor 31, as illustrated by the dotted line 62 will produce a suction and/or pushing effect inside the internal chamber 22 of the dental implant 10 so to refresh the interstitial fluids inside the internal channel 22. Most of the continuous glucose monitoring devices have delay compared to finger pricking measuring results. Refreshing the interstitial fluids inside the internal chamber will reduce this delay. The movement of the sensor 31 inside the chamber can be by a mechanical mechanism or activated by the patient with chewing, face muscle movements like cheeks and/or lips and/or by tongue movements. However, the delay, called also “lag time” between glucose levels in interstitial fluid and the blood is expected to be minimal if the sensor inside bone since the bone marrow tissue is actually like the blood. Therefore, placing a sensor inside bone is close to placing a sensor inside the blood. Minimal lag time is important for developing an “artificial pancreas” that includes an insulin pump requiring “real time” measurements.

In some embodiments, the protruding component can be temporarily removed for replacing the used analyte measuring component with a new analyte measuring component and then the protruding component can be connected again to protect the new analyte measuring component. This way, no surgical procedure is needed to replace the implantable analyte measuring component.

The device can include a semi-permeable barrier that can have several properties and can be in several locations at the device. The semi-permeable barrier can be flexible like a membrane or cloth and/or can be a more rigid structure. In some embodiments, the semi-permeable barrier may be porous and can include one or more of the following materials: nylon, cellulose, cellulose acetate, polypropylene, polyethylene, poly(ethylene terephthalate) (PET), poly(ether sulfone), poly(vinylidene difluoride) (PVDF), poly(tetrafluoroethylene) (PTFE), polyethylene glycol (PEG), polycarbonate, poly(oxazolines), poly(acrylamides), poly(electrolytes), poly(ethers), poly(vinyl pyrolidone), Poly(ethylenimines), poly(vinyl alcohol), poly(acrylates and methacrylates), poly(maleic anhydride), 2-hydroxyethyl methacrylate (HEMA), poly(ethylene glycol) methacrylate (PEGMA), and/or acrylic/methacrylic acid and any combination thereof. The semi-permeable barrier may be hydrophilic or amphiphilic.

In one embodiment illustrated in FIG. 7, a distal opening and/or a perforated region 70, for example a mesh, can be at the apical part 12 of the dental implant 12. This distal opening and/or perforated region 70 can have large holes that allow the entrance of cells. This distal opening and/or perforated region 70 can be covered by a semi-permeable barrier 71 separating between the jawbone and the internal chamber 22 of the dental implant 10. The semi-permeable barrier 71 can be permeable to an analyte and restrict passage therethrough of substances, e.g., cells and large molecules, which could potentially interfere with the measuring of the relevant analyte inside the internal chamber 22. The semi-permeable barrier 71 can be attached to the inner side of the dental implant 10 as illustrated in FIG. 7 and/or to the external surface of the dental implant and/or inside the distal opening 70. The semi-permeable barrier 71 can be designed with different pore sizes to enable certain molecules to enter the internal channel 22 from the tissue outside the dental implant 10 and to prevent cells and/or other molecules from entering inside the internal chamber 22.

The apical part of the dental implant can have inside a semi-permeable barrier and/or a tube shape semi-permeable barrier 81 as illustrated in FIG. 8A or a bag shaped semi-permeable barrier 82 as illustrated in FIG. 8B. The tube/bag shape semi-permeable barrier 81/82 can be attached to the inner walls of the dental implant for example by gluing and/or by a mechanical attachment. The tube/bag shaped semi-permeable barrier 81/82 can be inside the dental implant to surround the internal chamber 22 without being attached to the inner walls of the dental implant and/or to be connected to the dental implant in a detachable connection to enable replacement of the tube/bag shaped semi-permeable barrier 81/82. The replacement of the semi-permeable barrier can be useful in cases when the semi-permeable barrier is damaged during the insertion of the dental implant and/or during the insertion of the analyte measuring component inside the dental implant and/or when the semi-permeable barrier blocks completely over time the entrance of interstitial fluids and/or the analyte and/or when the semi-permeable barrier becomes distorted over time. The internal walls of the dental implant can have an undercut and/or a slot to fixate the semi-permeable barrier so the semi-permeable barrier will not move unintentionally coronally. The semi-permeable barrier 81/82 can be like an envelope of the internal chamber 22, so the walls of the internal chamber 22 are the semi-permeable barrier 81/82. The semi-permeable barrier 81 can be the analyte permeable region meaning to be all around permeable to the interstitial fluids and the analyte and to block entrance of cells or to have at least one region which is the analyte permeable region 16 as illustrated in FIG. 8B, while the rest of the semi-permeable barrier 81 can block the entrance of the interstitial fluids and the analyte. For example, the semi-permeable barrier 81/82 can be made from metal like titanium, palladium, platinum, tantalum, molybdenum, zirconium, biocompatible polymers and any combination thereof that blocks the entrance of interstitial fluids and have at least one analyte permeable region 16 that will enable the entrance of interstitial fluids and the analyte inside the internal chamber 22, which can be surrounded by the semi-permeable barrier 81/82 while preventing the entrance of cells inside internal chamber 22 surrounded by the semi-permeable barrier 81/82. The analyte permeable region 16 of the semi-permeable barrier 81/82 can be, for example, a semi-permeable membrane 83 connected to opening/s in the semi-permeable barrier 81/82. The connection of the semi-permeable membrane 83 to the opening/s in the semi-permeable barrier 81/82 can be from the outside and/or inside of the semi-permeable barrier 81/82.

The tube-shape semi-permeable barrier 81 can be in contact with the apical floor 84 of the dental implant 10 to prevent entrance of cells inside the internal chamber 22 from between the apical floor 84 and the apical end of the tube-shape semi-permeable membrane 81. To better prevent such entrance of cells, the apical floor 84 of the dental implant 10 can have a slot and the apical end of the tube-shape semi-permeable barrier 81 will be inside the slot at the floor 84 of the dental implant. The slot at the floor 84 of the dental implant can be for example circular and to be formed for example by a trephine drill. To better seal the internal chamber 22 from entrance of cells and from passage of bacteria, the semi-permeable barrier 81 can have an apical fixation. The apical part of the dental implant can have an apical fixating screw 86 that will be screwed to an apical internal thread 85 at the apical part of the dental implant. The apical fixating screw 86 can push the semi-permeable barrier 81 to be in contact with the internal walls of the apical part of the dental implant, apically to the distal opening/s 70 of the apical part of the dental implant. The head of the fixating screw 86 can be conical and the internal walls of the apical part of the dental implant adjacent the head of the apical fixating screw 86 can be also conical to better fixate the semi-permeable barrier 81 between the apical fixating screw 86 and the internal walls of the apical part 84 of the dental implant.

The semi-permeable barrier 81, 82 can have also a more coronal fixation. The dental implant can have a hollow fixating screw 88 that will be screwed to a coronal internal thread 87 at a more coronal region of the dental implant. The hollow fixating screw 88 can push the semi-permeable barrier 81 to be in contact with the internal walls of the dental implant, coronally to the distal opening/s 70 of the apical part of the dental implant. The head of the hollow fixating screw 88 can be conical and the internal walls of the apical part of the dental implant adjacent the head of the hollow fixating screw 88 can be also conical to better fixate the semi-permeable barrier 81 between the hollow fixating screw 88 and the internal walls of the dental implant. (Please note that in FIG. 8A the hollow fixating screws 86, 88, the semi-permeable membrane 81 and the internal walls of the dental implant are not touching for illustration purposes, while for fixation there is contact). The coronal internal thread 87 can continue coronally to the hollow fixating screw 88 to enable the fixation of other components like dental abutments and/or analyte measuring components by a screwed connection. The hollow fixating screw 88 being hollow to allow the insertion of component/s, like the sensor of the analyte measuring component inside the internal chamber surrounded by the semi-permeable barrier 81/82. The apical fixation screw 86 and/or the hollow fixation screw 88 prevent the entrance of bacteria and cells inside the internal chamber 22. The apical fixating screw 86 and/or the hollow fixating screw 88 can have a resilient band to be in contact with the semi-permeable barrier 81, 82 to prevent direct contact of the metal screw with the semi-permeable barrier that can damage the barrier during rotating the screws.

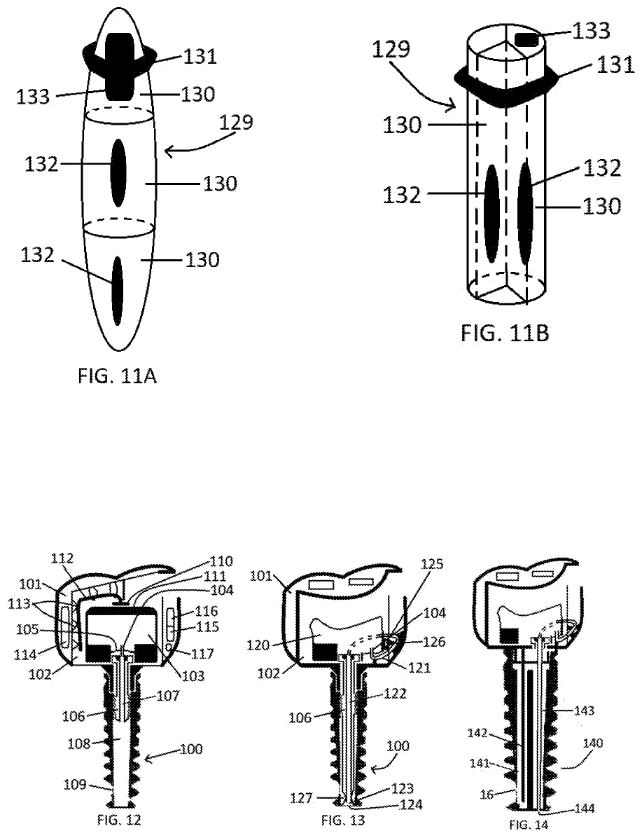

A space can be formed between the semi-permeable membrane 81, 82 and the walls of the dental implant so capillaries can enter this space to form a capillary chamber 27 as explained and illustrated in more detail hereafter in FIG. 10. This capillaries chamber can be at the apical end of the dental implant and/or adjacent the lateral walls of the dental implant and/or lateral walls of the semi-permeable membrane 81, 82.

The fixation of the semi-permeable barrier 81, 82 can be by other mechanisms. For example, by a click connection instead of a screw. For example, by a flexible ring/coil inside the semi-permeable barrier 81, 82 that will push the semi-permeable barrier 81, 82 inside a slot at the lateral walls of the dental implant. This flexible ring/coil stabilizes the semi-permeable barrier 81, 82 and also prevents the entrance of cells from the bone inside the internal chamber and prevents the passage of bacteria from the dental implant to the bone.

The semi-permeable membrane 83 and/or the tube/bag semi-permeable barrier 81/82 can be made from a guided bone regeneration membrane (G.B.R. membrane) known in the dental implantology field. For example, the semi-permeable membrane 83 and/or the tube/bag semi-permeable barrier 81/82 can be made from Polyurethane and/or Polyethylene Terephthalate and/or polytetrafluoroethylene (PTFE) membrane and/or expanded-polytetrafluoroethylene (ePTFE) membrane (for example, from Cytoflex membrane from Unicare Biomedical Inc., USA) and/or dense-polytetrafluoroethylene membrane (dPTFE) (for example, from DSI Israel implants, Israel or Cytoplast membranes from Osteogenics Biomedical Inc.). The PTFE/ePTFE/dPTFE membranes can be reinforced with titanium stripes (titanium reinforced PTFE membranes are also marketed by the above-mentioned companies and by other companies. The membranes can be reinforced with other rigid materials). The titanium reinforced membranes can be more easily inserted inside the dental implant, can be more stable, fixated and preserve their shape and not collapse compared to non-reinforced membranes. The semi-permeable barrier can be circular all around and formed by rolling a membrane. The semi-permeable barrier can be along only part of the internal walls of the dental implant and not to be in a full circular shape. The semi-permeable barrier can be supplied already as a tube, like artificial blood vessels (for example from: Maquet, Terumo, Gore, B. Braun, Bard, Jotec GmbH, LeMaitre Vascular, Perouse Medical and Nicast). The semi-permeable barrier can be impregnated with disinfecting materials and/or antibiotics.

The device can include an energy source 40, for example, a small battery that can be located in several positions. For example, the energy source 40 can be connected to the proximal region of the dental implant and/or it can be part of the protruding component, as illustrated in FIGS. 4, 5 and 6 and/or it can be part of the analyte measuring component as illustrated in FIG. 3. For example, the dental implant 10 can include a proximal external thread and the energy source 40 can include a matching internal thread so to be screwed to the dental implant. The energy source can be also connected to the dental implant like a healing cap so the dental implant 10 can include a proximal internal thread and the energy source can include a matching external thread so to be screwed to the dental implant through the proximal opening 21 of the dental implant.

In all the embodiments, the device can also include a mechanism that transforms movements into electrical energy. The mechanism can be for example similar to known mechanisms used in the mechanical watch field. The energy source can be also recharged using chewing movements and forces. In one embodiment, at least part of the protruding component can be flexible, and its movements caused by the tongue or chewing will be transformed to recharge the battery. The battery can be also charged by wireless transmission (for example by Bluetooth and/or WiFi). In another embodiment the protruding component 36 can include a spring and a coronal plate that will move when chewing. These movements can be used to recharge the battery. Such movements can be used also for activating a sensor in the analyte measuring component to perform measuring and/or to transmit a measuring result. For example, the patient can move with its tongue part of the protruding component to indicate starting of a measurement and/or transmitting the results to a receiving device. If the measuring mechanism is based on light the movement of the protruding component by the tongue can activate a light emitting component. The movements can be horizontal and/or vertical.

Such movements can be also used to move and/or recycle materials and/or fluids inside the internal chamber 22 and/or inside the protruding component 36 and/or inside the analyte measuring component 30. Such movements of materials and/or fluids can increase diffusion and passage of the analyte so to change the concentration of the analyte inside the device and/or adjacent the device to be closer to the concentration of the analyte in the body.

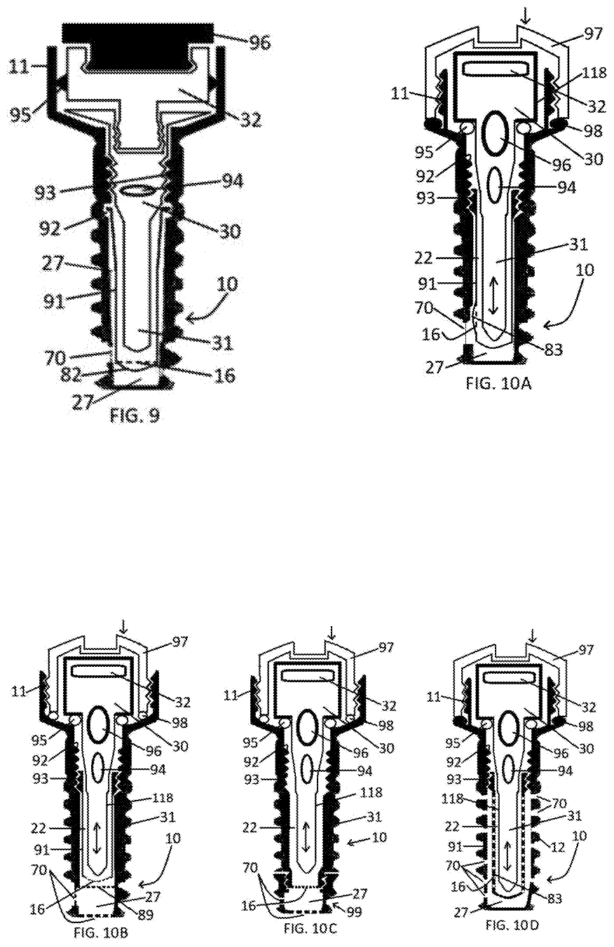

FIG. 9 illustrates an embodiment in which the dental implant 10 with a distal opening/s 70 has an internal semi-permeable barrier 91 in the shape of a bag adjacent the inner walls of the dental implant. The dental implant can have an internal slot 92 for the fixation of the semi-permeable barrier 91. The semi-permeable barrier 91 can have at least one analyte permeable region 16 that can include a semi-permeable membrane 83 covering opening/s in the semi-permeable barrier 91. The semi-permeable barrier 91 can include a small protrusion to enable easy catching, for example by a pinset, for insertion and/or removal. The dental implant can have an internal thread 93 that can be coronally to the semi-permeable barrier 91. The internal thread 93 can be used for the fixation of a dental component like an abutment. In this embodiment the internal thread 93 can be used for the fixation of the analyte measuring component 30. The sensor 31 can be inserted inside the bag shaped semi-permeable membrane 91. The analyte measuring component 30 can include a light emitting component 94. The analyte measuring component 30 can be connected to a transmitter 32. The connection can be in various ways, for example, by screwing the transmitter 32 that has an external thread to the internal thread inside the analyte measuring component 30. The transmitter 32 can have a resilient band 95 to be between the transmitter 32 and the walls of the coronal part 11 of the dental implant 10 to seal the coronal opening of the dental implant. The transmitter 32 can be connected to an energy source 96 by various connections to enable easy replacement and/or charging of the energy source 96. For example, by screwing the energy source to the transmitter 32 or by a click mechanism using the flexibility of part of the transmitter 32 and/or the energy source 96, as illustrated in FIG. 9. The location of the transmitter 32 and energy source 96 of FIG. 9 can be replaced so the energy source will be connected to the analyte measuring component and the transmitter 32 will be coronally to the energy source to enable better transmission.

A space can be formed between the semi-permeable membrane 91 and the walls of the dental implant so capillaries can enter this space to form a capillary chamber 27 as explained and illustrated in more detail hereafter in FIG. 10. This capillaries chamber can be at the apical end of the dental implant and/or adjacent the lateral walls of the dental implant and/or lateral walls of the semi-permeable membrane 91.

FIG. 10A illustrates an embodiment in which the dental implant 10 has at least one distal opening and/or a perforated region 70 and has an internal semi-permeable barrier 91 in the shape of a bag adjacent the inner walls of the dental implant. The semi-permeable barrier 91 can have at least one analyte permeable region 16 or can be entirely permeable to the analyte while blocking the passage of some larger molecules. The analyte permeable region 16 of the semi-permeable barrier 91 can have opening/s covered by a semi-permeable membrane 83. The at least one distal opening and/or a perforated region 70 can allow the entrance of small blood vessels like capillaries to create a capillaries chamber 27 inside the dental implant. The capillaries chamber 27 can be apically to the semi-permeable barrier 91 and/or around it. The openings at the perforated region 70 can have several optional dimensions. For example, the openings at the perforated region 70 can be large enough to allow the entrance of blood vessels, for example the diameter of the hole/perforations of the perforated region 70 can be 100-900 micrometers and/or 200-700 micrometers and/or 300-600 and/or to have a diameter larger than 1 mm. For example, the openings at the perforated region 70 can be large enough to allow the entrance of blood vessels and small enough to prevent the formation of dense bone tissue and/or dense connective tissue. For example, the diameter of the perforations of the perforated region 70 can be of 1-20 micrometers and/or 5-10 micrometers and/or 10-20 micrometers and/or 20-30 micrometers and/or 30-50 micrometers and/or 5-40 micrometers and/or 30-60 micrometers and/or 40-80 micrometers and/or 50-100 micrometers and/or 40-150 micrometers and/or 5-100 micrometers and/or 20-80 micrometers and/or 30-100 micrometers. There can be many combinations of pore shape and interconnectivity of the pores to enhance capillaries/blood vessels formation and entrance inside the capillaries chamber 27. For example, to have holes larger than 1 mm at one region and smaller holes at another region so capillaries can enter the capillaries chamber 27 through the large holes and exit the capillaries chamber 27 through the smaller holes to have blood circulation through the capillary chamber 27. The dental implant can have several perforated regions 70, while each perforated region 70 has the same diameter of holes or a different diameter of holes to influence the entrance and exit of capillaries inside and outside the capillaries chamber. For example, a first perforated region with the larger holes can be more apically to a second perforated region with smaller holes. For example, a first perforated region with the larger holes can be more coronally to a second perforated region with smaller holes. For example, a first perforated region with the larger holes can be on one side of the dental implant while a second perforated region with smaller holes being on the other side and any combination of the locations and/or holes properties of the perforated regions. The density of the perforations at the perforated region 70 and/or pore shapes can influence also the entrance and/or exit of blood vessels and/or capillaries inside and outside the capillaries chamber and the formation of bone and/or connective tissues inside the capillaries chamber 27. For example, the pores can occupy 10-20% and/or 20-30% and/or 30-40% and/or 40-50% and/or 50-60% and/or 60-80% and or 30-90% of the surface of the perforated region 70. The pores can have several shapes like round and/or oval and/or rectangular and/or hexagonal and/or any other shape.

The capillaries chamber 27 can have an internal surface that enhance capillary formation and/or entrance. For example, the internal surface of the capillaries chamber inside the dental implant can include materials that enhance angiogenesis. For example, various active molecules like growth factors and/or cytokines like VGF, VEGF, VEGF-2, VEGF-165, rhVEGF. antimicrobial peptide (AMP), (TNF)-α, TGF-β1, TGF-β, prostaglandin I-2 (PGI-2), VEGF-A, eNOS, iNOS, fibroblast growth factors (FGF)-1, (FGF)-2, and epidermal growth factor (EGF), EGF, hepatocyte growth factor (HGF), bone morphogenic proteins (BMPs like BMP-2, BMP-4), insulin-like growth factors (IGF-1 and 2), platelet-derived growth factor (PDGF), interleukin IL-1β and IL-6, monocyte chemoattractant protein, lipopolysaccharide (LPS), (MCP)-1, macrophage inflammatory protein (MIP)-1α, fibronectin, ROS (reactive oxygen species). The internal surface of the capillaries chamber can be rough surface that enhance angiogenesis, for example, S.L.A. and/or S.L.A. Active surfaces from Straumann having pore sizes of 20-40 micrometers and inside pore sizes of 2-4 micrometers. The internal surface of the capillaries chamber can have nano-scale roughness or other means to increase wettability and/or to be more hydrophilic. This can be done for example by acid etching the surface in addition to sandblasting or alone. Sodium or potassium hydroxide can be used on the surface followed by heat treatment. The result of this treatment is the formation of titanium hydroxide (Ti—OH) on the surface of implant, which increases the charge of the surface and adhesion of proteins. The surface can be treated with hydrogen peroxide (H2O2), acids, alkali, anodization, chemical vapor deposition, and sol-gel. The surface can be treated by grit blasting, which can be done by titanium or aluminum particles ranging in size between 25-75 micrometers. The sandblasting and acid-etching treatment methods can include the grit blasting with 250-500 micrometers size particles, and the acid-etching can be done with hydrochloric and sulfuric acids. The surface can be treated by anodizing and/or oxidizing. The surface can be coated with hydroxyapatite and/or include various drugs and antibiotics like simvastatin, Gentamycin and/or Tetracycline-HCl. The dental implant can have an internal thread 92 for the fixation of the semi-permeable barrier 91. The semi-permeable barrier 91 can include an external thread 93 and an anti-rotational element to enable easy insertion and removal of the semi-permeable barrier 91 by screwing. Other means of insertion and/or connection of the semi-permeable barrier 91 can be used. Removal of the semi-permeable barrier 91 may be needed after several months/years in case the semi-permeable barrier 91 becomes perforated with too larger holes or blocks the entrance of the analyte and/or becomes distorted and/or prevents the insertion of the analyte measuring component 30. The internal thread 92 of the dental implant 10 can continue coronally to the semi-permeable barrier 91. The internal thread 92 can be used for the fixation of a sensor and/or dental abutment and/or crown and/or bridge and/or denture and/or any other dental component. In this embodiment the analyte measuring component 30 can be inserted inside the dental implant 10 so the sensor 31 can be inserted inside the semi-permeable barrier tube/bag 91. The analyte measuring component 30 can include a light emitting component 94. The analyte measuring component 30 can include a transmitter 32. The analyte measuring component 30 can have a resilient band 95 to be between the analyte measuring component 30 and the walls of the coronal part 11 of the dental implant 10 to prevent bacteria from the oral cavity to reach the internal chamber 22 of the dental implant 10. The semi-permeable barrier bag/tube 91 can have a resilient band (not shown) to be between the analyte measuring component 30 and the walls of the apical part of the dental implant to seal the internal chamber 22 of the dental implant 10. The semi-permeable barrier tube/bag 91 can have a tapered region to be in contact with a compatible tapered region inside the dental implant 10 to seal the internal chamber 22 of the dental implant 10. The analyte measuring component 30 can include an energy source 96, for example a battery. A sealing/closing cap 97 can be connected to the coronal part 11 of the dental implant 10. The connection can be for example by a click and/or by screwing. For example, the coronal part 11 of the dental implant can have an external thread and the closing cap 97 can have an internal thread as illustrated in FIG. 10A and/or the coronal part 11 of the dental implant can have an internal thread and the closing cap 97 can have an external thread. The coronal part 11 can further have a resilient band 98 to be in contact with the closing cap 97 to seal the connection between the closing cap 97 and the coronal part 11 of the dental implant 10. The closing cap 97 can be made from a flexible material like PEEK, silicone, PVC, thin metals etc. Preferably the closing cap 97 can be made from materials that will enable free transmission of the transmitter 32 to the receiver (not shown). The flexible closing cap 97 can be bended towards the dental implant 10 when chewing on the closing cap 97 to move the analyte measuring component 30 inside the dental implant 10. Such movements of the analyte measuring component 30 can trigger activation of the analyte measuring component 30 and/or movements of the interstitial fluids inside the internal chamber 22 of the dental implant from outside the dental implant and/or from the capillaries chamber 27. The analyte measuring component 30 can move back to its initial position by a spring (not shown) and/or by the resilient band 95 and/or by being connected to the flexible closing cap 97, which returns to its initial position and/or any other mechanism. When the analyte measuring component 30 needs to be replaced the sealing closing cap 97 can be removed, a new analyte measuring component 30 can be inserted and the closing cap 97 can be placed again over the new analyte measuring component 30. This replacement procedure can be done every several days and/or months and/or years without any surgery.

The analyte measuring component 30 and/or some of its components can include a capsule and/or envelope 118, which can surround the analyte measuring component 30 and/or some of its components. The capsule/envelope 118 can be inserted inside the internal channel of the dental implant. The analyte measuring component 30 can be inside the capsule/envelope 118 and/or part of the capsule/envelope 118 and/or part of the capsule/envelope and/or part of the analyte measuring component 30. The capsule/envelope 118 can be made at least partially from a semi-permeable barrier or without such a barrier. The inside of the envelope and/or the capsule/envelope can include the measuring mechanism and/or the transmitting mechanism and/or the energy source and/or any other components of the device. The capsule/envelope can be part of the sensor, for example, to use fluorescent glucose-indicating polymer technology. The capsule/envelope 118 can be fixated inside the internal channel of the dental implant by a snap connection and/or screwed connection and/or any other connection. The connection can be a sealed connection.

FIG. 10B illustrates an embodiment which is similar to the embodiment of FIG. 10A. In this embodiment, the coronal part 11 of the dental implant can have an internal thread and the closing cap 97 can have an external thread so the closing cap 97 can be screwed inside the coronal part without contact with the gums around the dental implant. The dental implant can have, in addition to the distal opening and/or perforated region 70, a second semi-permeable barrier and/or mesh 89, which can be less permeable than the perforated region 70. The second semi-permeable barrier/mesh 89 can be attached to the apical part of the dental implant while the analyte permeable region 16 can be part of the of the analyte measuring component and/or part of the capsule/envelope 118 and/or can be replaceable with the analyte measuring component. The analyte permeable region 16 and the second semi-permeable barriers 89 can have similar porosities to enable the passage of similar analytes or can be different, so one semi-permeable barrier allows the passage of less molecules than the other semi-permeable barrier. For example, the second semi-permeable barriers 89, can be more permeable than the analyte permeable region 16. The second semi-permeable barrier 89, which can be fixated or not fixated to the dental implant can serve to prevent penetration of cells and blood vessels inside the internal channel, since such penetration can interfere with the replacement of the capsule 118 and/or the analyte measuring component and/or materials and/or components of the device. For example, the second semi-permeable barrier 89 can allow the penetration of only interstitial fluids and several molecules inside the chamber while the analyte permeable region 16, which can be part of the analyte measuring component and/or part of the capsule 118, can allow the passage of only small molecules like the analyte. This way, when the capsule 118 is replaced it can be inserted inside the internal channel or chamber of the dental implant having interstitial fluids inside, without direct contact with the tissues around the dental implant. The capsule 118 can be easily inserted because there are no cells insides the internal chamber and there is no encapsulation reaction to the capsule material.

In the embodiment of FIG. 10B, there can be also a capillaries chamber 27, having the distal opening/s 70 at least in one region of the boundaries of the capillaries chamber 27 and the second semi-permeable barriers 89 at at least one another region of the boundaries of the capillaries chamber 27. Capillaries can be inside the capillaries chamber 27 without bone and dense connective tissue to enable the presence of interstitial fluid representing the blood concentration of the analyte with reduced delay.

FIG. 10C illustrates an embodiment which is similar to the embodiments of FIGS. 10A and 10B. In this embodiment the apical part of the implant can be made from two parts which are connected to each other. The connection can be by screwing and/or gluing and/or friction and/or other known methods for connecting parts. The most apical part 99 of the dental implant can have inside the capillaries chamber 27. In this configuration it can be easier to treat the surface of the internal walls of the capillaries chamber 27 to enhance the entrance of capillaries inside. The surface treatment/s can be any treatment as discussed above for example by adding materials that promote angiogenesis or producing rough internal surface. In this embodiment the most apical part can have the distal opening/s 70 and the more coronal part of the apical part can have the analyte permeable region 16, which can be fixated to the implant or can be detachable as illustrated for example in FIGS. 10A and 10B.

FIG. 10D illustrates an embodiment which is similar to the embodiments of FIGS. 10A, 10B and 10C. In this embodiment the distal opening/s are not only at the distal region of the apical part 12 of the dental implant, but along the apical part 12 of the dental implant. The analyte measuring component 30 can be covered by a semi-permeable membrane 83 to form the analyte permeable region along and/or around the analyte measuring component 30. In this configuration the capillaries chamber 27 can be present around the analyte measuring component 30 and not only adjacent the distal end of the dental implant. In this configuration the surface of the capillaries chamber in contact with the internal chamber 22 of the dental implant can be larger and therefore the diffusion of the analyte from the capillaries chamber inside the internal chamber 22 can be faster and the delay regarding the blood concentration of the analyte can be shorter. The analyte permeable region 16 and/or semi-permeable membrane 83 can be in a sleeve/tube shape as described before and can be replaceable as described before.

In all the embodiments the system/device and/or the jawbone implant and/or the analyte measuring component and/or analyte permeable region/semi-permeable barrier/analyte permeable membrane can include additional membrane that prevents clogging of the analyte permeable region/semi-permeable barrier/analyte permeable membrane. The surrounding environment of the jawbone implant can include materials like proteins and/or lipid that can aggregate/precipitate on the analyte permeable membrane and/or attached to the analyte permeable membrane. Since the analyte permeable membrane can have very small pores, for example, pores having a diameter in the range of few nanometers and even less than a nanometer, these pores can be clogged or blocked resulting in interfering with the entrance of the glucose/analyte. This can cause delay in the measurements, non-accurate measurements, and even non-function of the device. One solution is the replacement of the analyte measuring component with the analyte permeable membrane or replacing only the analyte permeable membrane if the analyte measuring component (sensor) is still functioning. To enable replacement of only the analyte permeable membrane, this membrane needs to be easily accessible from the oral cavity and easily replaced. Another solution is to have an additional membrane—an anti-clogging membrane located laterally/peripherally to the analyte permeable membrane. This anti-clogging membrane can be designed to be easily replaced when this anti-clogging membrane becomes clogged/blocked while keeping the sensor inside the jawbone implant. This anti-clogging membrane can be the same as the analyte permeable membrane or can have pores with a different diameter, for example, larger pores that allow the passage of the analyte while blocking the entrance of materials that can block the analyte permeable membrane. The anti-clogging membrane can surround the sensor and/or to be between the capillary chamber and the internal chamber with the sensor.

A capillaries chamber can be in all the embodiments of the present application. The capillaries chamber can have several separate perforated regions to enhance the entrance of capillaries from the jawbone inside the capillaries chamber. More than one perforated region can allow the entrance of capillaries from one perforated region and the exit of capillaries from another perforated region so capillaries can pass through the capillary chamber. These perforated regions of the capillaries chamber 27 can have pores of the same sizes or each perforated region having pores of different sizes. In another embodiment at least one of the perforated regions of the capillaries chamber can be covered by a resorbable barrier that will enable the entrance and/or exit of capillaries inside and/or outside the capillaries chamber 27 at a later stage.

The current implantable devices having capsules from fluorescent glucose-indicating polymer technology to measure glucose in the interstitial fluid (like Everesense from Sensonics Inc., U.S.) can remain inside the tissue only for several months and requires a small surgery of removal and another small surgery for insertion of a new capsule about every three months. In the innovative devices of the current application, a similar capsule can be inserted inside the internal channel of the dental implant and replaced without any surgery at all, since the dental implant can be inside the tissue and protruding to oral cavity for many years, without encapsulation reaction. In addition, implantable capsules based on fluorescent glucose-indicating polymer technology requires a light source which is attached to the skin and therefore limit the patient and needs to be replaced and/or recharged very often. In the innovative devices of the current application, the light source can be also inside the mouth. The light source can be part of the protruding component. The light source can be connected to other teeth and/or be part of a dental crown, a dental bridge, a denture, a plate and any appliance that can be inside the mouth, permanently or removably. The capsule can have a semi-permeable barrier and can be without such a barrier to allow direct contact of the envelope of the capsule with the surrounding interstitial fluids.

There are measuring mechanisms which are activated by light in a special wavelength. The protruding component can be transparent or partially transparent to transmit light inside the internal chamber of the dental implant where the analyte measuring component can be located. The protruding component and/or the dental implant inside the bone can be made for example from zirconium to enable light transmission inside the internal chamber of the dental implant. The light can originate from a light source located outside the mouth. For example, a light source from a smartphone device with a dedicated application, which can be activated by the patient or another person when the patient is opening his mouth. The light source can transmit light indirectly through tissue, for example, a light source, which transmits light through the cheeks and/or lips. The light source can be for example, a small flashlight, which is adjacent the face, for example by being part of eyeglasses and/or earrings and/or hearing aid devices. The light source can be inside the mouth as explained above as part of the device or in another location in the mouth.

In some embodiments the pores of the analyte permeable region/semi-permeable barrier allow the passage of glucose. In another embodiments the pores of the analyte permeable region/semi-permeable barrier allow the passage of cholesterol and/or triglycerides and/or ferritin and or other metabolites in the interstitial fluids.

In other embodiments different analyte permeable region/semi-permeable barrier are adjacent different openings at the apical part of the dental implant. For example, a first analyte permeable region/semi-permeable barrier that allows the passage of a first analyte adjacent a first opening and a second analyte permeable region/semi-permeable barrier that allows the passage of a second analyte adjacent a second opening.

In another embodiment, a capsule 129 with an analyte permeable region/semi-permeable barrier can be divided into two or more sub-chambers 130 as illustrated in FIGS. 11A and 11B. The sub-chambers 130 can be arranged along the coronal-apical axis as illustrated in FIG. 11A or around the coronal-apical axis as illustrated in FIG. 11B. The sub-chambers 130 can be also arranged around a central sub-chamber or any other arrangement of sub-chambers inside the capsule 129 that will be inserted inside the dental implant. In each chamber or sub-chamber 130, a sensor 132 can be inserted, while the sensors in each chamber can be different. The capsule can have a resilient ring 131 to be in contact with the inner walls of the coronal part of the dental implant to seal the coronal part of the dental implant.

In several embodiments each sub-chamber has a different analyte permeable region that allows the passage of a different analyte. In one embodiment into each sub-chamber 130 a different sensor can be inserted. For example, into a chamber that allows the entrance of glucose a sensor for glucose can be inserted and into a chamber that allows the entrance of triglycerides a sensor for triglycerides can be inserted.

In several embodiments several sub-chambers 130 can allow the entrance of the same analyte, for example glucose, and into each sub-chamber a different sensor 132 can be inserted that measure the concentration of this analyte. As explained above there are several methods to measure the concentration of glucose. Each method has its advantages, disadvantages and range of inaccuracy. By having several sensors with different results, a computerized system 133 can be used for example to average the results or to ignore unreasonable results. By doing so the transmitted result will be more accurate and the system will have less false alarms. The different sensors 132 can have also different life cycles and times of accuracy and delays. For example, one sensor can be accurate only after several weeks after insertion while another sensor can be accurate at the beginning and to lose its accuracy over time. A computerized system 133 can predict the accurate glucose concentration from the input received from these two sensors, while considering the time elapsed since the insertion of each sensor inside the dental implant.

In another embodiment, a capsule can be located at the apical part of the dental implant and to continue to the coronal part and/or even outside the dental implant. For example, the coronal region of the capsule that can be protruding outside the dental implant towards the oral cavity can be partially or fully translucent. The capsule can have sensitive molecules that will react differently to different glucose concentration. The sensitive material can be exposed to different energy sources like light in various wave lengths and/or sounds in various wave lengths including ultrasounds and/or magnetic forces and/or electric currents and/or electromagnetic technologies and/or thermal modulations. The sensitive material can react differently to the energy sources depending on the glucose concentration or the energy source will react differently depending on the state of the sensitive material and/or enzyme.

The capsule can also have a capsule envelope material that can be a measuring material and/or a sensitive material and/or contain fluid with an analyte sensitive material and/or allow the entrance of the analyte inside the capsule. The capsule can be also at least partially protruding to the oral cavity. The various energy sources and sensors can be applied on these capsule materials. For example, illuminating a transparent and/or translucent capsule and analyzing the light reflection which can indicate the concentration of the glucose/analyte inside the capsule. The device can include a coronal sealing element that will protect the capsule and seal it from the oral cavity. The coronal sealing element can be the protruding component or part of it, and it can be connected to the dental implant in variable connections, preferably sealed connections. The coronal sealing element can be translucent and/or transparent.

At least part of the apical part of the dental implant can be made from semi-permeable material/s, for example semi-permeable titanium and/or semi-permeable zirconium (and/or from other materials like platinum, palladium, tantalum, molybdenum, zirconium, biocompatible polymers and any combination thereof), while this part of the apical part can be porous to enable the passage of an analyte while preventing the passage of other larger molecules or to be porous to enable entrance of interstitial fluids while preventing entrance of cells and blood vessels. Such a semi-permeable titanium and/or zirconium can be made for example by 3D printing of titanium and/or zirconium, for example by using titanium and/or zirconium powder and soldering the powder using lasers to form the dental implant or using high temperature to modify the titanium and/or zirconium structure. Other materials like palladium and/or platinum etc. can be also used. Another optional method to perforate the walls of the apical part of the dental implant can be by using lasers preferably along a thin region of the walls of the apical part of the dental implant. In this method as well as in other methods the number of pores and the size of the pores can be controlled by the intensity, wavelength and duration of the laser.

The apical part and the coronal part of the dental implant can be separated and sealed from each other. By doing so, other components can be attached to the coronal part of the dental implant, while maintaining the sterility of the internal chamber inside the apical part of the dental implant. For example, dental abutment and/or transducer and/or transmitter and/or battery. The sealing between the apical part and the coronal part of the dental implant can be a resilient biocompatible material like silicone and/or Teflon and/or polymers like PEEK and/or other biocompatible materials. The sealing element can be also a screw or screw having a resilient band made for example from the materials listed above.

The apical part of the dental implant can have a transparent and/or translucent region. The transparent/translucent region can be also perforated to allow the passage of the analyte so to serve as the analyte permeable region. The transparent/translucent region of the apical part of the dental implant can be sealed while an analyte permeable region can be located at another location of the apical part of the dental implant. The apical part of the dental implant with the transparent/translucent region can be sealed to prevent passage of materials inside the dental implant. In this configuration the measurements can be done using a light emitting measuring device without direct contact with the analyte. The transparent/translucent region can be made from several materials, for example Zirconium, glass, PEEK, polyurethane, plastic and/or any biocompatible polymer. The measuring device or sensor can utilize for example fluorescent, glucose indicating polymer technology to measure glucose in the interstitial fluid. The design of such a dental implant and analyte measuring component can be similar to the design illustrated in FIGS. 1-10, while instead of having an analyte permeable region having a transparent region and the device further includes a light source.

In another embodiment the content of the capsule and/or internal chamber of the dental implant can be replaced. For example, a sensing glucose material can be replaced every several days or/and weeks or/and months. It is also possible to replace the entire capsule including the semi-permeable material, if present, every several days or/and weeks or/and months. The replacement of the capsule and/or its content and/or the content of the internal chamber of the dental implant can be done for example by opening and/or removing the coronal sealing element of the dental implant that will allow access to the internal chamber of the dental implant. After the replacement of the capsule and/or the content of the internal chamber of dental implant, the coronal sealing element can be placed again to seal the coronal opening of the dental implant and protect the capsule and/or internal chamber. The coronal sealing element can be a dental abutment and/or a healing abutment and/or a crown and/or a transducer and/or transmitter and/or receiver and/or battery.

The coronal part of the dental implant can include a sealing element that will prevent bacteria from entering the internal channel. The energy source can function as a sealing element. The sealing element can include a resilient band or O-ring, for example from silicone, nitrile, rubber, latex, polycarbonate and/or P.T.F.E, so it will be in contact with the dental implant and the sealing element.

In another embodiment, the analyte measuring element can include a resilient band or O-ring so it will be in contact with the dental implant and/or the sealing element. The device can include several sealing elements.

In another embodiment the coronal region of the capsule can allow taking samples of the fluid inside the capsule. For example, the capsule can include a valve that will release a small drop when force is activated on the coronal region of the capsule. In another embodiment a needle can be inserted through a self-sealing region inside the capsule to take a fluid sample. The capsule can also include a one-directional valve.

The device can be also connected to a treating and/or injecting device. For example, if the device is measuring glucose levels and the glucose levels are high, the device can transmit a signal to activate an insulin pump. This way the entire system functions as an artificial pancreas. The system measures the glucose levels and inject insulin when needed. In case the glucose levels are too low, the receiving device can alert the patient and/or alert other personnel like medical staff and/or family members of the patient.

The medicine and/or insulin pump (for example, MiniMed 670 G from Medtronic Inc.) can have a receiver that will activate the medicine/insulin pump to deliver the medicine and/or insulin inside the body according to the measuring results received form the analyte measuring component. The medicine and/or insulin pump can be connected to the abdomen and/or arm and/or other locations regularly used for insulin pumps.

In some embodiments the medicine and/or insulin pump can be inside the mouth connected to a dental implant 100 as illustrate in FIG. 12. The dental implant connected to the medicine and/or insulin pump can be the same dental implant, which is connected to the analyte measuring component or a separate dental implant. The dental implant 100 can be connected to a dental crown 101, which can be opened to enable access to the inside space 102 of the dental crown 101. A reservoir 103 like a small ampule with insulin can be inserted inside the inside space 102 of the dental crown 101. The dental crown 101 can be closed in a sealed manner so the reservoir 103 can be inside the dental crown 101 while saliva and bacteria are prevented from entering inside the inside space 102 of the dental crown 101. The dental crown 101 can include a needle 104 that can penetrate a flexible region 105 of the reservoir 103 when the reservoir 103 is inserted inside the inside space 102 of the dental crown 101. The needle 104 can be part of a fixating screw 106 that fixate the dental crown 101 to the dental implant 100. The fixating screw 106 can be hollow having an internal channel 107 to lead from the needle 104 to an inside chamber 108 of the dental implant 100. The dental implant 100 can have an opening 109 that will connect the inside chamber 108 of the dental implant 100 and the tissue adjacent the dental implant. The opening 109 can be at a side wall of the dental implant and/or at the apical end of the dental implant. The opening 109 can be completely open or can have a semi-permeable barrier that will allow the release of the insulin and/or medicine to the tissue while preventing the entrance of cells and blood vessels inside the dental implant 100.

The dental crown 101 can include a pump or injecting mechanism that can include a piston 110 pushing the upper part 111 of the reservoir 103. The piston 110 can be connected to a flexible wire 112 that can be moved by a mechanical component 113, which can be activated by chewing or by an energy source like a battery 114. The dental crown 101 can include a receiver 115 that will receive the glucose levels transmissions and a computerized system 116 that will activate the mechanical component 113 of the injecting mechanism to inject the insulin from the reservoir 103 inside the dental implant 100 and to the tissue according to the glucose level received.

The computerized system 116 can alert the patient that no insulin is left in the reservoir 103 and replacement is needed. The alert can be by a transmitter 117 to an outside receiver like a smartphone and/or by vibration and/or sound from the dental crown 101. The patient and/or other personnel can then open the crown 101, take out the empty ampule 103 and insert a new ampule. The frequency of replacements can be dependent on the patient's condition. Some patients will need several replacements a day while other will replace the ampule every several days. The frequency of replacing the ampules will probably be higher than the frequency of replacing an ampule in a conventional insulin pump, since the volume of the intra-oral capsule is smaller. However, the replacement of the oral ampule 103 is much simpler and painless while the replacement of an ampule in a conventional pump is more complicated and requires inserting a needle inside the body. Moreover, a patient with an intra-oral pump and ampule is not limited in his activities and no one can see the pump, as is the case with conventional insulin pumps.

The dental implant with the insulin pump can be the same dental implant with the analyte measuring component. In this case the hollow fixating screw 106 in FIG. 12 can be the sensor, the opening 109 can be like the analyte permeable region of FIGS. 1-10 to allow the entrance of the analyte and interstitial fluids, the passage of the insulin outside to the tissue while preventing the entrance of cells and blood vessels and passage of bacteria.

In another embodiment, the insulin pump can be based on the mechanical principles of a peristaltic pump. A peristaltic pump is a type of positive displacement pump used for pumping a variety of fluids. In the embodiment of FIG. 13 the insulin can be contained within a reservoir like a flexible container 120, like a bag, which can be fitted inside the inside space 102 of a dental crown 101. The dental crown 101 can be fixated to the dental implant 100 for example by a hollow fixating screw 106. A needle 104 can be inserted inside the flexible container 120. A flexible tube 121 connects the needle 104 and the hollow fixating screw 106 to enable the advancement of the insulin from the flexible container 120 through the inner space 122 of the hollow fixating screw 106 inside a medicine inner chamber 123 located inside the dental implant 100 adjacent an opening 124 in the dental implant's walls and/or apical end. The insulin can then enter to the adjacent tissue through the opening 124 of the dental implant 100. The opening 124 can be completely open or to have a semi-peimeable barrier that allows the release of the insulin while preventing the entrance of cells from the tissue inside the dental implant. A rotor 125 with a number of “rollers”, “shoes”, “wipers”, or “lobes” 126 attached to the external circumference of the rotor 125 compresses the flexible tube 121 against a surface, for example, an inner surface of the dental crown 101. As the rotor 125 turns, the part of the flexible tube 121 under compression is pinched closed (or “occludes”) thus forcing the insulin to be pumped to move through the flexible tube 121. Additionally, as the flexible tube 121 opens to its natural state after the passing of a roller 126 (“restitution” or “resilience”) fluid flow is induced from the flexible container 120 inside the flexible tube 121. Typically, there will be two or more rollers, or wipers 126, occluding the flexible tube 121, trapping between them a body of fluid. The body of fluid is then transported by pressure toward the hollow fixating screw 106. The peristaltic pump can be indexed through partial revolutions to deliver smaller amounts of insulin according to the glucose levels.

The dental implant 100 of FIG. 13 with the peristaltic insulin pump can be the same dental implant with the analyte measuring component. In this case the hollow fixating screw 106 in FIG. 13 can be the sensor, the opening 124 can be like the analyte permeable region of FIGS. 1-10 to allow the entrance of the analyte and interstitial fluids, the passage of the insulin outside to the tissue while preventing the entrance of cells and passage of bacteria. The medicine inner chamber 123 for the insulin can be a separate chamber from the internal chamber 127 for measuring the analyte or the two inner chambers can be the same chamber. In most of the embodiments the jawbone implant with the analyte measuring component will not be the jawbone implant with the insulin pump since the injection of insulin adjacent the sensor might change the glucose levels adjacent the sensor.

In another embodiment in which the same dental implant being connected to both the insulin pump and the analyte measuring component, as illustrated in FIG. 14, the dental implant 140 can have two separate internal chambers, a first internal chamber 141 for the sensor 142 and a second internal chamber 143 for the insertion of the medicine/insulin. The second internal chamber 143 for the medicine/insulin can have a medicine/insulin permeable region 144 that can allow the passage of insulin while preventing the passage of other molecules. The medicine/insulin permeable region 144 can be a hole with a diameter of an insulin needle having an inner diameter of approximately 0.1-0.5 mm or larger than 0.5 mm. In this embodiment the insulin pump can be for example a peristaltic pump as explained for FIG. 13. The implant can have in addition to the medicine/insulin permeable region 144 an analyte permeable region 16 adjacent the sensor 142.

The reservoir of the medicine/insulin can be part of a dental prosthesis, for example a crown, a bridge, a bar and/or a denture. The reservoir can be filled every several hours/days by injecting inside the reservoir inside the mouth or the reservoir can be taken out of the mouth and filled outside the mouth. In another embodiment, the container and/or reservoir with the medicine/insulin and the pump can be for example at a buccal and/or lingual flange of a denture and can include a tube leading to the dental implant. The reservoir can also include disinfecting materials.

The device that includes also the medicine/insulin pump can function as an artificial pancreas, while the patient doesn't need to insert needles and sensors inside the body every several days. If the sensor is accurate enough, also finger pricking can be eliminated. The insulin pump can also have a transmitter that will indicate the amount of insulin in the reservoir and the status of the pump.

The insulin/medicine reservoir can be connected to the dental implant in a click connection or any other connection. For example, a first half of a dental crown connected to the dental implant can include the transmitter of the analyte measuring component, while the second half of the dental crown can include the medicine/insulin reservoir and the pump. In this embodiment the second half-dental crown with the insulin reservoir can be replaced or filled with insulin every several hour/days while the first half-dental crown connected to the sensor and/or transmitter remains in place for several weeks/months.

The insulin reservoir can be an implanted reservoir, for example, the reservoir can be placed inside the maxillary sinus and can be connected to a dental implant in the maxillary alveolar ridge below the maxillary sinus or in the zygoma bone that will allow filling the reservoir with the insulin. The reservoir can be placed above the Schneiderian membrane or below the Schneiderian membrane to be between the floor of the maxillary sinus and the Schneiderian membrane.

The bone implant can be placed adjacent the ear and the reservoir will be connected to the ear like a hearing device and/or eyeglasses.

In all the embodiments, the reservoir can be an expandable reservoir. For example, it can be inserted inside the maxillary sinus through the alveolar ridge in a procedure resembling a closed sinus lift procedure. The reservoir will be inserted in a small dimension and expanded after the insertion. The reservoir can be made from a variety of biocompatible materials. It can be made from rigid materials like titanium and/or flexible materials like silicone, nylon, Teflon etc.

The measuring result can be recorded and/or transmitted to a receiving device. The receiving device can be for example a smartphone having an application for storing and analyzing the transmissions of the device. The transmission can be continuous or every predetermined time interval. The transmission can be activated by the patient, for example, by applying force to a protruding part of the device, for example by chewing and/or using the tongue. The transmission can be activated by light and/or sound and/or voice. The transmission can be activated by the receiving device, which can also transmit signals to the analyte measuring component.

The sensor can also transmit alarms when measurements are out of predetermined thresholds. For example, when glucose levels and/or oxygen saturation and/or body temperature and/or blood pressure and/or heart rate are too high or too low and/or any other measurement is too high or too low, the device can send an alarm signal to the receiving device, which can send an alarm to the patient or other personnel. The device can also alert the patient when the receiving device like a smartphone is not near the patient, by movements, sound, light and electrical current to the gums and/or bone causing some sensation to the patient. The sensor can also transmit signals that will allow locating the location of the patient in cases of emergency.

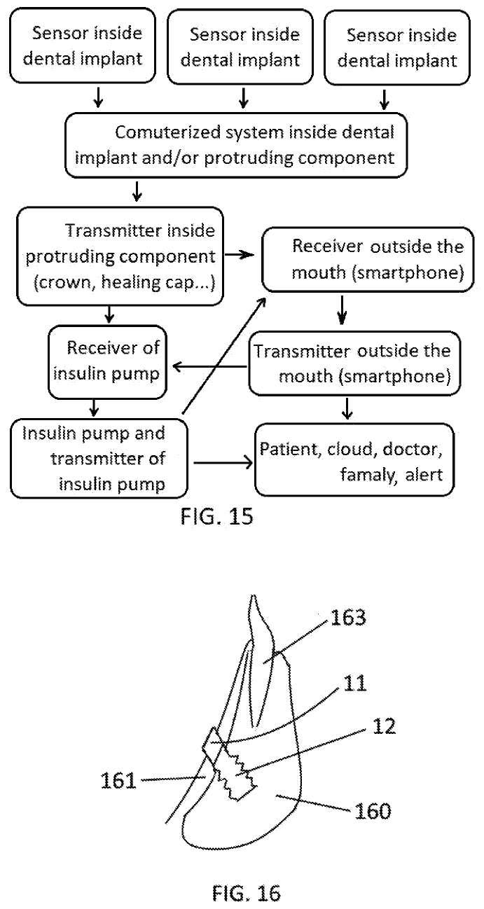

FIG. 15 illustrates a block/flow diagram of an embodiment of the device. The device can include one or more sensors to measure the analyte in various methods and/or locations. The sensors can be inside the dental implant and/or in contact with the inside of the dental implant. The results of the sensor/s are delivered to a computerized system that can calculate the analyte concentration based on the results of the sensor/s. The computerized system can be inside the dental implant and/or inside the protruding component and/or in another location in the mouth or outside the mouth. The result of the computerized system can be delivered to a transmitter that can be inside the dental implant and/or inside the protruding component and/or in another location in the mouth or outside the mouth.

The transmitter transmits the result to a receiver or several receivers. The receiver/s can be inside the dental implant and/or inside the protruding component and/or in another location in the mouth or outside the mouth. The receiver can be, for example, inside a smartphone and/or a dedicated device and/or an insulin pump. The receiver/s can deliver the result to a memory storage device (not illustrated) to record the result. The memory storage device can be part of the smartphone and/or dedicated device. The receiver can deliver the result to a transmitter that can be part of the smartphone and/or dedicated device that will transmit the result to remote memory storage device (like a cloud) and/or to other devices to inform the patient or other personnel. In case the result is received by an insulin pump, then a computerized system inside the pump decides if to inject and how much insulin to inject. The insulin pump can be inside the mouth or outside the mouth. The insulin pump can have a transmitter to transmit the amount of insulin injected and the time of injection and in addition the amount of insulin left in the reservoir. The transmission of the insulin pump can be to the receivers and/or memory storage devices mentioned above and/or other receivers and memory storage devices.

The system can include sensors and devices known in the IoT (Internet of Things) field to communicate between the components of the system.

In some applications of the present invention, the inner space of the dental implant contains an optically-transparent and glucose-permeable material, e.g., a gel or polymer, configured to define the sampling region. Typically, the analyte permeable region/semi-permeable barrier can be configured to restrict passage, into the sampling region, of cells and some molecules having a molecular weight greater than the molecular weight of the analyte configured to be measured by the device. In some applications of the present invention, the sampling region comprises genetically-engineered cells that produce a protein that is able to bind with the analyte and to undergo a conformational change in a detectable manner. Alternatively, the protein can be placed in the sampling region without cells. The optical measuring device detects the conformational change, via a signal generated indicative of a level of the analyte in the subject. The signal itself is embodied as the amount of light of different wavelengths emitted by the protein. Typically, but not necessarily, FRET techniques—i.e., Forster Resonance Energy Transfer, also known as fluorescence resonance energy transfer, are used to detect the conformational change. These genetically-engineered cells and/or protein may be used in combination with the detection methods described in the present application.

The analyte permeable region/semi-permeable barrier and the material inside the dental implant can be designed such that the glucose inside the dental implant can be generally in equilibrium with the body interstitial level of glucose or alternatively, with the level of glucose in the jaws.

The dental implant can have at least one external thread and an anti-rotational element for rotating and inserting the dental implant inside the jawbone. The anti-rotational element can be internal and/or external. The anti-rotational element can have a polygonal configuration like hexagon, square, rectangular, octagon, triangular etc. and any polygonal shape and/or any other configuration known in implant dentistry and/or to have vertical and/or horizontal indentations or any form which is not circular. The anti-rotational element can be internal—inside the dental implant and/or external at the coronal part of the dental implant.

The dental implant can be a bone level implant or can be like a tissue level dental implant. In a tissue level dental implant, the coronal end of the dental implant is placed outside the bone protruding to the gingiva and even protruding to the oral cavity. It is easier to replace the analyte measuring element and/or material inside the dental implant and/or the energy source with a tissue level implant and to prevent saliva and other oral fluids from entering the internal chamber of the dental implant.

The dental implant can include more than one anti-rotational element, so any combinations of the above mentioned anti-rotational elements and other anti-rotational elements can be in several locations along the dental implant. The coronal part of the dental implant can have also an internal thread for the connection of a healing abutment and/or a dental abutment and/or a dental crown and/or a bridge and/or the analyte measuring component. The use of a dental crown can allow the use of larger components, like a larger battery and/or light source and/or a larger reservoir of sensing solutions and/or a larger analyte measuring component. Therefore, the protruding component can be any dental prosthesis component like a crown, bridge, denture etc.

The dental implant can have rings instead of a thread or can be without external protrusion so it will be inserted by pushing instead of screwing.

The dental implant can be a custom-made dental implant using CAD/CAM technology or 3D technology to fit the perfect implant for each patient, especially for patients that don't have an available place for conventional dental implants.

The coronal part of the dental implant or the implant head can have different diameter or outline than the body of the implant and can have an undercut and/or intrusions and/or a slot 150 as illustrated in FIGS. 1 and 2 to enable attachment of a clamp for holding a rubber dam, similarly to the placement of a rubber dam on natural teeth when performing root canal therapy. The rubber dam can be placed to prevent saliva entering the internal chamber of the implant when replacing elements or materials inside the internal chamber of the dental implant. The device can be part of a kit that includes a dedicated protecting element that can be fixated by the undercut 150. The slot 150 can be designed to enable the attachment of a rubber dam without the use of a clamp. Such a clamp and a rubber dam can be produced in special size and configuration to be easily attached to the coronal part of the dental implant. The depth of the undercut 150 towards the central axis of the dental implant can be 0.2 mm-1.5 mm or 0.4-1.0 mm or 0.7-1.2 mm. Since the rubber dam can't completely guarantee that no bacteria will enter the internal chamber of the dental implant, the dental implant can include a chamber for disinfecting material.

The external surface of the dental implant can influence the bone to implant contact (B.I.C.). Rough surfaces, for example, SLA (from Straumann Holding AG, Switzerland) or TiUnite (from Nobel Biocare AG, Switzerland) increase the B.I.C. compared to machine surface, which was the surface of the first Branemark implants. Polished surface reduces the B.I.C. Therefore, the apical intra-bony part of the dental implant of the present invention can have several regions with different roughness. The apical intra-bony part of the dental implant can have regions with machine surface and/or regions with polish surface and/or regions with rough surface. The rough surface regions will eventually be adjacent a denser bone tissue to increase the stability of the dental implant, while the machine surface and/or the polished surface will be adjacent a less dense tissue with higher concentration of blood vessels and/or capillaries and allow for more accurate measurements. In some embodiments, the external surface of the apical part of the dental implant adjacent the analyte permeable region can have regions with different roughness. For example, a distal opening of the dental implant and/or perforated region of the dental implant and/or the region with the semi-permeable barrier and/or the analyte permeable region of the apical part of the dental implant can be adjacent a polish surface or surrounded by a polished surface and/or adjacent a machine surface and/or surrounded by a machine surface so as to increase the interstitial fluids adjacent these parts of the implant. In another embodiment the distal opening of the dental implant and/or perforated region of the dental implant and/or the region with the semi-permeable barrier of the dental implant and/or the analyte permeable region of the dental implant can be adjacent a rough surface or surrounded by a rough surface. The external surface of the apical part of the dental implant can have a first roughness adjacent the place where interstitial fluids enter inside the apical part of the dental implant (the distal opening of the dental implant and/or a perforated region of the dental implant and/or the a region with the semi-permeable barrier of the dental implant and/or the analyte permeable region of the dental implant) and second roughness at other regions of the external surface of the apical part of the dental implant, the second roughness being larger than the first roughness.

The implant can be placed in various locations. For example: It can be placed in the place of missing tooth, after extracting a tooth, distally to the location of the teeth, in the ramus, in the retro-molar region in the mandible, in the tuberosity in the maxilla, in the pterygoid region, in the Zygoma bone, in the palate, between roots of teeth, in the symphysis, in the anterior lingual side of the mandible, bellow the lower incisors and/or canines. In the anterior buccal side of the mandible, bellow the lower incisors and/or canines, below the Schneiderian membrane of the maxillary sinus and/or the nose and in every location where orthodontic implants are placed, for example: Facial surface maxillary/mandibular alveolar ridge mesial to 1st molar and/or 2nd premolar, maxillary subANS region, parasagittal midpalate, zygomatic buttress, infrazygomatic crest or posterior lateral palate; mandibular ascending ramus, external oblique ridge and buccal shelf. The buccal shelf has several advantages since it usually has enough bone for implants, allows easy insertion of the implants, allows easy replacement of sensors by the patient while minimally inconvenience for the patient.

The dental implant can be placed so part of it is inside bone and part of it is inside the soft tissue. For example, the analyte permeable region can be adjacent the soft tissue for faster insertion of the analyte inside the internal chamber of the dental implant to reduce the delay between the blood concentration of the analyte and the analyte concentration inside the dental implant. The dental implant can be partially inside the gingiva and/or mucosa. The dental implant can be at least partially inside and/or adjacent the nasopalatine canal and/or incisive canal and/or greater palatine artery canal. The dental implant can be lingual to the upper central incisors adjacent the nasopalatine canal surrounded by bone or partially inside the nasopalatine canal and/or to be adjacent blood vessels, arteries and/or veins, for example branches of the palatine artery and/or facial artery and/or lingual artery.

Placing the jawbone implant below the anterior incisors is easier for the dentist to insert the implant and for future replacements of the analyte measuring component. There is also a blood vessel at the middle of the mandible coming from the lingual side. This location is also more convenient for activating, transmitting and communicating with external devises like a smartphone.

Placing the jawbone implant in the various locations, like below the anterior incisors might require placing the implant in various angulations to the bone surface. Therefore the top of the coronal part of the jawbone implant can be a sloped coronal part similar to the coronal part of the—OsseoSpeed® Profile EV implant from Dentsply-Sirona Inc. Similar dental implant is described in U.S. Pat. No. 9,271,812 B2 to Richard Cottrell. However, the jawbone implant can be a tissue level implant with a sloped coronal edge so the sloped coronal part has a Smoove external surface. FIG. 16 illustrates an embodiment of the jawbone implant inside the mandibular bone 160 bellow the lower incisor 163. The coronal part 11 of the jawbone implant protrudes through the mucosa 161 lining the mandibular bone 160. The coronal edge of the coronal part 11 being sloped or angled to the longitudinal axis of the jawbone implant. The coronal part can be at least partially smooth and the apical edge of the smooth coronal part can be sloped or angled to the longitudinal axis of the jawbone implant regardless if the coronal edge of the coronal part being also sloped. In case the coronal edge and apical edge of the smooth coronal region are both angled to the longitudinal axis of the jawbone implant, the coronal edge and the apical edge of the smooth coronal part can have different angulation and different morphologic.

The implant can be placed outside mouth, for example adjacent the ear and/or the nose. The dental implant can be inserted using a guided surgery stent and/or in a flapless procedure.

In all the embodiments, the dental implant can be part of the analyte measuring component. The material of the dental implant can be part of the sensor and/or be used for the transmission of electrical current between elements of the analyte measuring component.

In all the embodiments of the invention the connection between the various elements (tubes, connectors, cannula, injecting element, sensors, transmitters, crowns etc.) can be by several options, for example Luer connection, screwed connection, friction connection, soldering, gluing, connection through additional connectors or adaptors and any combination thereof, etc.

The components of the system can be made from a variety of materials used in the medical field and are not limited to special materials or group of materials. For example, from metals and/or plastics, for example stainless steel and/or titanium and/or ceramics and/or nylon and/or silicone. The components of the system can be made also from materials that are for implantation and also from bio-dissipative material. The device can include also bioactive materials.

Although the present invention has been described and illustrated in the context of certain embodiments, it will be understood that modifications may be made without departing from the spirit of the invention.

What is claimed is:

1. A jawbone implant for monitoring human body analyte comprising:
a coronal part of said jawbone implant and an apical part of said jawbone implant, said apical part of said jawbone implant has an internal chamber and a capillaries chamber, said capillaries chamber has at least one perforated wall having at least one perforation of a diameter of at least 5 micrometers that enables entrance of capillaries from a jawbone inside said capillaries chamber when said apical part of said jawbone being inside a jawbone, said jawbone implant has further at least one analyte permeable region between said capillaries chamber and said internal chamber configured so when said apical part of said jawbone implant being inside said jawbone, interstitial fluids and said analyte can enter from said capillaries chamber inside said internal chamber through said at least one analyte permeable region, a largest diameter of a perforation in said analyte permeable region being smaller than 1 micrometer to prevent cells to enter from said capillaries chamber inside said internal chamber through said at least one analyte permeable region, said internal chamber being accessible from said coronal part of said jawbone implant to enable insertion of an analyte measuring component inside said internal chamber through said coronal part of said jawbone implant to measure said analyte inside said internal chamber.

2. The jawbone implant of claim 1, wherein said at least part of said capillaries chamber being laterally to said internal chamber.

3. The jawbone implant of any one of claims 1-2, wherein said analyte permeable region being perforated, a largest diameter of a perforation in said analyte permeable region being smaller than 0.3 micrometer.

4. The jawbone implant of any one of claims 1-3, wherein said analyte permeable region being perforated, a largest diameter of a perforation in said analyte permeable region being smaller than 0.1 micrometer.

5. The jawbone implant of any one of claims 1-4, wherein at least part of said capillaries chamber being at an apical end of said apical part of said jawbone implant.

6. The jawbone implant of any one of claims 1-5, wherein said jawbone implant being part of a device that includes said analyte measuring component.

7. The jawbone implant of any one of claims 1-6, wherein said at least one analyte permeable region being at a wall of said apical part of said jawbone implant.

8. The jawbone implant of any one of claims 1-7, wherein said at least one analyte permeable region being located at a distal end of said apical part of said jawbone implant.

9. The jawbone implant of any one of claims 1-8, wherein said at least one analyte permeable region being inside said apical part of said jawbone implant.

10. The jawbone implant of any one of claims 1-9, wherein said at least one analyte permeable region being part of a semi-permeable barrier located inside said apical part of said jawbone implant.

11. The jawbone implant of any one of claims 1-10, wherein said apical part of said jawbone implant being perforated to enable entrance of said interstitial fluids and said analyte from said jaw inside said apical part of said jawbone implant, an envelope being inside said apical part of said jawbone implant, said at least one analyte permeable region being part of said envelope so said internal chamber being at least partially surrounded by said envelope to prevent entrance of cells inside said internal chamber.

12. The jawbone implant of claim 11, wherein said envelop has a tube shape.

13. The jawbone implant of any one of claims 11-12, wherein said envelop has a bag shape.

14. The jawbone implant of any one of claims 1-13, wherein said capillaries chamber surrounds said internal chamber.

15. The jawbone implant of claim 1-14, wherein said perforated wall of said capillaries chamber that enables entrance of capillaries has several pores having a diameter of more than 100 micrometers.

16. The jawbone implant of any one of claims 1-15, wherein said capillaries chamber has rough internal surface.

17. The jawbone implant of any one of claims 1-16, wherein said capillaries chamber has a material that promotes angiogenesis.

18. The jawbone implant of any one of claims 1-17, wherein perforated wall of said capillaries chamber has a first region and a second region, a diameter of holes at said first region of said wall of said capillaries chamber being different than a diameter of holes at said second region of said wall of said capillaries chamber.

19. The jawbone implant of any one of claims 11-18, wherein said envelop being fixated to the internal walls of said jawbone implant by at least one screw.

20. The jawbone implant of any one of claims 11-19, wherein an apical region of said envelop being fixated to the internal walls of said jawbone implant by an apical fixating screw, a coronal region of said envelop being fixated to the internal walls of said jawbone implant by a hollow fixating screw.

21. The jawbone implant of any one of claims 1-20, wherein a wall of said internal chamber has at least one distal opening that allows entrance of said interstitial fluids and said analyte inside said internal chamber, said at least one distal opening being covered by a semi-permeable membrane that allows passage of said interstitial fluids and said analyte from said jaw inside said internal chamber while preventing passage of cells from said jaw inside said internal chamber.

22. The jawbone implant of any one of claims 1-21, wherein a wall of said internal chamber being a wall of an envelope inside said apical part of said jawbone implant.

23. The jawbone implant of any one of claims 1-22, wherein a majority of the walls of said internal chamber are a semi-permeable barrier located inside said jawbone implant.

24. The jawbone implant of any one of claims 1-23, wherein said coronal part of jawbone implant has a proximal opening, said analyte measuring component being at least partially inserted inside said internal chamber through said proximal opening of said coronal part of said jawbone implant.

25. The jawbone implant of any one of claims 1-24, wherein said analyte measuring component being connected to said jawbone implant in a sealed manner to prevent bacteria from entering from said oral cavity inside said internal chamber.

26. The jawbone implant of any one of claims 1-25, wherein at least part of said analyte measuring component being inside said internal chamber.

27. The jawbone implant of any one of claims 1-26, wherein said coronal part of said jawbone implant being connected to a protruding component that protrudes to the oral cavity.

28. The jawbone implant of claim 27, wherein said at least part of said analyte measuring component being inside said protruding component.

29. The jawbone implant of any one of claims 27-28, wherein said jawbone implant and said protruding component are one-piece.

30. The jawbone implant of any one of claims 27-28, wherein said jawbone implant and said protruding component are connected in a detachable connection.

31. The jawbone implant of any one of claims 27-30, wherein said protruding component being wider than said jawbone implant.

32. The jawbone implant of any one of claims 27-31, wherein said protruding component protrudes buccally from said jawbone implant to be at the buccal vestibulum.

33. The jawbone implant of any one of claims 27-32, wherein said protruding component function as a jawbone prosthesis selected from the group consisting of: a crown, a bridge, a denture, an abutment, a supra-structure, an infra-structure and any combination thereof.

34. The jawbone implant of any one of claims 27-33, wherein said protruding component being at least partially translucent to enable light from said oral cavity to enter said internal chamber.

35. The jawbone implant of any one of claims 27-34, wherein said protruding component has at least one undercut for fixation of a rubber dam.

36. The jawbone implant of any one of claims 27-35, wherein said protruding component includes an energy source being charged by chewing on said protruding component and/or by the tongue pushing said protruding component.

37. The jawbone implant of any one of claims 27-36, wherein said protruding component includes a transducer that transduce a measuring result of said analyte measuring component to a receiving component, said transducer being activated by chewing on said protruding component and/or by the tongue pushing said protruding component.

38. The jawbone implant of any one of claims 27-37, wherein said protruding component being configured to move by chewing over said protruding component and/or by the tongue pushing said protruding component and further configured to activate said analyte measuring component to measure said analyte when said protruding component being moved.

39. The jawbone implant of any one of claims 27-38, wherein at least part of a light emitting component being located inside said protruding component that emits light inside said internal chamber to activate said analyte measuring component.

40. The jawbone implant of any one of claims 1-39, wherein at least part of a light emitting component being located inside said internal chamber.

41. The jawbone implant of any one of claims 1-40, wherein said jawbone implant being at least partially translucent to enable light from the oral cavity to enter said internal chamber.

42. The jawbone implant of any one of claims 1-41, wherein a circulating element being inside said jawbone implant to move fluids inside said internal chamber.

43. The jawbone implant of any one of claims 1-42, wherein said analyte measuring component being connected to said jawbone implant by a flexible connector to enable movements of said analyte measuring component inside said internal chamber during mastication.

44. The jawbone implant of any one of claims 1-43, wherein said apical part of said jawbone implant has at least one rough external surface region to promote bone tissue attachment to said rough external surface region and at least one smooth external surface region to prevent attachment of bone tissue to said smooth external surface region, said smooth external surface region being adjacent said at least one analyte permeable region.

45. The jawbone implant of any one of claims 1-44, wherein said apical part of said jawbone implant has a first rough external surface region with a first roughness to promote bone tissue attachment to said first rough external surface region and a second rough external surface region with a second roughness to promote attachment of bone tissue to said second rough external surface region, said first roughness being larger than said second roughness, said second rough external surface region being adjacent said at least one analyte permeable region.

46. The jawbone implant of any one of claims 1-45, wherein an external surface of said apical part includes a material that promotes angiogenesis.

47. The jawbone implant of any one of claims 1-46, wherein said internal chamber includes a measuring solution forming a chemical reaction with said analyte while said analyte measuring component being configured to measure said chemical reaction.

48. The jawbone implant of any one of claims 1-47, wherein said jawbone implant has a core and at least one external thread extending along at least part of said core.

49. The jawbone implant of any one of claims 1-48, wherein said analyte selected from the group consisting of: glucose, cholesterol, lipids, iron, ferritin, Natrium, potassium, salts, immunoglobulins, oxygen saturation, liver enzymes, hormones and any combination thereof.

50. The jawbone implant of any one of claims 1-49, wherein part of said jawbone implant being part of said analyte measuring component.
